(19) United States
(12) Patent Application Publication
(10) Pub. No.: US 2021/0353483 A1
(43) Pub. Date: Nov. 18, 2021

(54) FOLDABLE ISOLATION SYSTEM AND METHOD OF USE

(71) Applicant: Prep Tech, L.L.C., Westlake, LA (US)

(72) Inventors: Mark R. Moore, Westlake, LA (US); Peter Prados, Lafayette, LA (US)

(73) Assignee: Prep Tech, L.L.C., Westlake, LA (US)

(21) Appl. No.: 17/302,232

(22) Filed: Apr. 27, 2021

Related U.S. Application Data
(63) Continuation-in-part of application No. 17/301,698, filed on Apr. 12, 2021.
Application No. 17/301,698 is a continuation-in-part of application No. 17/301,217, filed on Mar. 29, 2021.
(60) Provisional application No. 63/008,331, filed on Apr. 10, 2020.
(60) Provisional application No. 62/704,093, filed on Apr. 21, 2020.
(60) Provisional application No. 63/001,379, filed on Mar. 29, 2020.
(60) Provisional application No. 62/704,092, filed on Apr. 21, 2020.
(60) Provisional application No. 62/704,192, filed on Apr. 27, 2020.
(60) Provisional application No. 62/704,385, filed on May 7, 2020.
(60) Provisional application No. 62/705,297, filed on Jun. 20, 2020.
(60) Provisional application No. 62/706,393, filed on Aug. 13, 2020.

(51) Int. Cl. A61G 10/00; A61G 10/02; A61B 46/20; A61B 90/00; A61B 90/40

(52) U.S. Cl. CPC A61G 10/005; A61G 10/023; A61B 90/40; A61B 90/05; A61B 46/20

(57) ABSTRACT

A medial isolette is provided. A foldable box-type enclosure and base rail type enclosure are provided which supplies an economical and easily assembled isolation chamber. A flexible drape is provided to further isolate the patient and effectively control a negative pressure environment within the isolette. Access sports with integrated or removable gloves are provided to access the patient when the isolette is in use. A component access panel is provided for ducted connection of patient gas circuits and leads.

### CROSS-REFERENCE TO RELATED APPLICATIONS

This application is a continuation-in-part of U.S. application Ser. No. 17/301,698 filed on Apr. 12, 2021, which claims priority benefit from U.S. Provisional Application No. 63/008,331, filed on Apr. 10, 2020; and U.S. Provisional Application No. 62/704,093, filed on Apr. 21, 2020, which is a continuation-in-part of U.S. application Ser. No. 17/301,217, filed on Mar. 29, 2021, which claims priority benefit from U.S. Provisional Application No. 63/001,379, filed on Mar. 29, 2020, and U.S. Provisional Application No. 62/704,092, filed on Apr. 21, 2020. This application claims priority benefit from U.S. Provisional Application No. 62/704,192, filed on Apr. 27, 2020; U.S. Provisional Application No. 62/704,385, filed on May 7, 2020; U.S. Provisional Application No. 62/705,297 filed on Jun. 20, 2020 and U.S. Provisional Application No. 62/706,393 filed on Aug. 13, 2020. The patent applications identified above are incorporated here by reference in their entirety to provide continuity of disclosure.

### FIELD OF THE INVENTION

The present invention relates to medical isolettes.

### BACKGROUND OF THE INVENTION

Patient isolation represents one of several measures that can be taken to control the spread of infection and highly contagious diseases. The goal of patient isolation is to contain airborne contagions produced by the patient, or alternatively, to limit airborne contagions to which the patient is exposed.

The U.S. Centers for Disease Control and Prevention (CDC) issues, and periodically revises, recommendations for various levels of patient isolation for disease control. Isolation is typically recommended when a patient is known to have a highly contagious viral or bacterial illness.

Special isolation protocols are used in the management of patients in isolation. Typical isolation protocols include mandating personal protective equipment such as gowns, masks, and gloves and applying physical controls, such as negative pressure rooms, laminar air flow and structural barriers. Negative pressure is used to reduce risk of exposure to airborne contagions produced by the patient.

Patients can produce airborne contagions in any number of circumstances. For example, airborne contagions are typically produced by a patient during intubation. Intubation is required when the patient cannot maintain their airway or cannot breathe without assistance. Intubation requires inserting an endotracheal tube, through the mouth and then into the airway. The tube is then connected to a ventilator. Intubation typically takes place after a patient is anaesthetized. Even so, patient reflex reactions usually cause coughing during the intubation procedure which can result in a significant amount of airborne contagion being released.

The requirement for patient isolation and intubation occurs in many circumstances. For example, patient isolation and intubation can take place in an emergency situation in the field. Emergency isolation in the field is difficult because physical control protocols such as negative pressure rooms and structural barriers are difficult to erect quickly and at low cost. Further exacerbating the problem, intubation is typically prescribed only once a dangerous condition is recognized. Oftentimes, such diagnoses are made in emergencies with extremely limited time to react.

Emergency situations, such as epidemics, also create special problems. For example, epidemics, such as swine flu, MERS-COV, Ebola and COVID-19 are considered highly contagious. Such highly contagious diseases certainly require effective negative pressure isolation and many times also require patient intubation, simultaneously. Effective negative pressure containment is critical to control rapid transmission of these diseases. In these situations, it is extremely difficult to protect caregivers during the intubation process, given the current state of the art in isolation barriers.

The prior art has attempted many different methods and structures to solve the problems of simple, inexpensive and portable isolation barriers but all have fallen short.

For example, U.S. Pat. No. 5,832,919 issued to Kano discloses a portable enclosure system which provides a suitable breathing atmosphere within a chamber, by use of positive pressure provided by fans and exhaust ports. However, the chamber does not allow access to the patient to provide medical treatment and fails to provide a negative pressure environment.

U.S. Pat. No. 7,757,689 issued to Chang discloses an inflatable isolation “cabin” for isolation of the entire patient, prior to transport. However, the cabin must be removed to provide medical treatment such as surgery or intubation.

Another example is disclosed in U.S. Pat. No. 4,949,714, to Orr. Orr discloses a medical hood for fitting over the head of a patient. A gas port leads to the interior of the hood for supplying respiratory gas. An outlet connects the interior of the hood and the ambient air for discharge of gas. However, the hood of Orr is rigid and therefore does not provide for a collapsible frame for ease of use and transport.

Another example is U.S. Pat. No. 5,950,625 to Bongiovanni, et al. Bongiovanni discloses an isolation bag for isolating a casualty from a contaminated environment. The bag is fabricated from a transparent biochemically resistive material and includes a self-contained transportable life support system. However, the transportable system, including the bag, is difficult to replicate cheaply or transport easily and is time consuming to assemble and use.

A need exists to provide a device and method to isolate a patient while allowing the patient to be an intubated or receive other medical treatment. Further, a need exists for an isolation chamber that may be quickly deployed to protect health care providers from contagions while completing the intubation procedure. Still further, a need exists for an isolette that is inexpensive and which can be easily and quickly mass produced to aid in mass isolation of patients with highly contagious epidemic diseases.

A further need exists for an isolette that provides a negative pressure environment to protect health care workers when accessing a patient.

### SUMMARY OF THE INVENTION

A preferred embodiment includes a semi-rigid panel die-cut in a specific design that can be folded along predesignated lines to create a sealed medical isolette. A flexible drape is attached to the isolette to enclose the upper torso and isolate a patient in the interior of the isolette where negative pressure is applied. In one form, the isolette is constructed from a bottom panel, two side panels, a front panel, and a top panel that are all connected by fold creases. The front panel accommodates two sealed access ports which allow access to the interior of the isolette. A pressurization port is provided for connection to a filtered negative pressure turbine. A component access panel is optionally provided which serves as a pass through for ventilation tubes and other various leads.

In a preferred embodiment, the side panels and top panel include transparent windows for viewing the patient.

In a preferred embodiment, the access ports can further include integrated gloves, resealable flaps, sealing rings, or iris diaphragms that cover the access ports. In another preferred embodiment, the access ports can include integrated or removable gloves fixed by separable fittings and collars into the access ports.

In one preferred embodiment, the component access panel includes removable plugs of various sizes that are fixed to the access panel by microperforated lines. When the plugs are removed, passages of various sizes are created that accommodate tubes and electrical leads.

In another preferred embodiment, the component access panel is provided with various connectors that accommodate a ventilation circuit, a Luer Lock connector or other connectors for a CO₂ monitor.

Another preferred embodiment of an isolette includes two semi-rigid panels, one of which may be separated into two separate pieces. A first panel is separated by fold lines into a bottom panel, a top panel, and two side panels. The first panel further includes a removable brace. A second panel creates the top panel. The top panel includes a removeable window which is covered by the drape. Each of the three pieces is folded into a set of shapes that, when assembled, form the isolette.

Another preferred embodiment also includes a semi-rigid panel die-cut in a specific design that can be folded along predetermined lines to encapsulate a supporting stainless steel frame. The supporting frame is secured in a stainless steel base tray which provides structural rigidity to the isolette. A drape is attached to the top panel and the side panels to effectively isolate the patient.

The drape subassembly is constructed from cost effective polyethylene sheeting. The drape includes an optional flexible seal and a strap for securing the drape around the patient.

In use, the isolette is assembled from a set of flat component parts which may be easily and quickly assembled. A separate drape is attached to the isolette, once assembled, to provide an ideal negative pressure environment for patient isolation. The patient is positioned inside the isolette and the drape is extended along the patient's torso. The flexible seal is positioned across the patient and secured by the strap. Access to the patient is provided by the gloves sealed into conveniently positioned access ports.

### BRIEF DESCRIPTION OF THE DRAWINGS

In the detailed description of the preferred embodiments presented below, reference is made to the accompanying drawings. Unless otherwise specified, all tolerances are ±20%.

FIG. 1 is an isometric view of a preferred embodiment of an isolette.

FIG. 2 is a plan view of a preferred embodiment of a base frame subassembly.

FIG. 3 is a plan view of a preferred embodiment of a drape subassembly.

FIG. 4A is an exploded isometric view of the preferred embodiment of an isolette.

FIG. 4B is a flowchart of assembly of a preferred embodiment of an isolette.

FIG. 5 is a detail view of an alternate preferred embodiment of a side panel.

FIG. 6 is a detail view of an alternate preferred embodiment of a side panel.

FIG. 7A is an exploded view of a preferred component access panel.

FIG. 7B is an exploded view of an alternate embodiment of an access panel.

FIG. 8 is an isometric view of an alternate embodiment of a component access panel.

FIG. 9A is an exploded isometric view of a preferred glove connection.

FIG. 9B is an isometric view of a preferred glove connection.

FIG. 9C is an isometric view of a preferred glove connection.

FIG. 9D is an exploded isometric view of a preferred glove connection.

FIG. 9E is an exploded isometric view of a preferred barrier cap.

FIG. 10 is a side view of a preferred embodiment of an isolette in use.

FIG. 11 is an isometric view of a preferred embodiment of an isolette.

FIG. 12 is a plan view of a preferred embodiment of a base frame subassembly.

FIG. 13 is a plan view of a preferred embodiment of a top subassembly.

FIG. 14 is a plan view of a preferred embodiment of a drape subassembly.

FIG. 15 is an exploded isometric view of a preferred embodiment of an isolette.

FIG. 16 is a flowchart of a preferred method of assembly of an isolette.

FIG. 17 is an isometric view of a preferred embodiment of an isolette.

FIG. 18 is an isometric view of a preferred embodiment of a base subassembly.

FIG. 19 is a detail view of a preferred base subassembly.

FIG. 20 is an isometric view of a preferred support frame subassembly.

FIG. 21 is a plan view of a preferred enclosure subassembly.

FIG. 22 is a plan view of a preferred drape subassembly.

FIG. 23A is an exploded isometric view of a preferred enclosure subassembly.

FIG. 23B is an exploded isometric view of a preferred embodiment of an isolette.

FIG. 23C is an isometric detail view of a preferred enclosure subassembly.

FIG. 23D is an isometric detail view of a preferred enclosure subassembly.

FIG. 23E is an isometric detail view of a preferred enclosure subassembly.

FIG. 23F is an isometric detail view of a preferred enclosure subassembly.

FIG. 24A is a flowchart of assembly of a preferred embodiment of an enclosure subassembly.

FIG. 24B is a flowchart of assembly of a preferred embodiment of an isolette.

FIG. 25 is a flowchart of a preferred method of use of an isolette.

### DETAILED DESCRIPTION OF THE INVENTION

In the description that follows, like parts are marked throughout the specification and figures with the same numerals, respectively. The figures are not necessarily drawn to scale and may be shown in exaggerated or generalized form in the interest of clarity and conciseness.

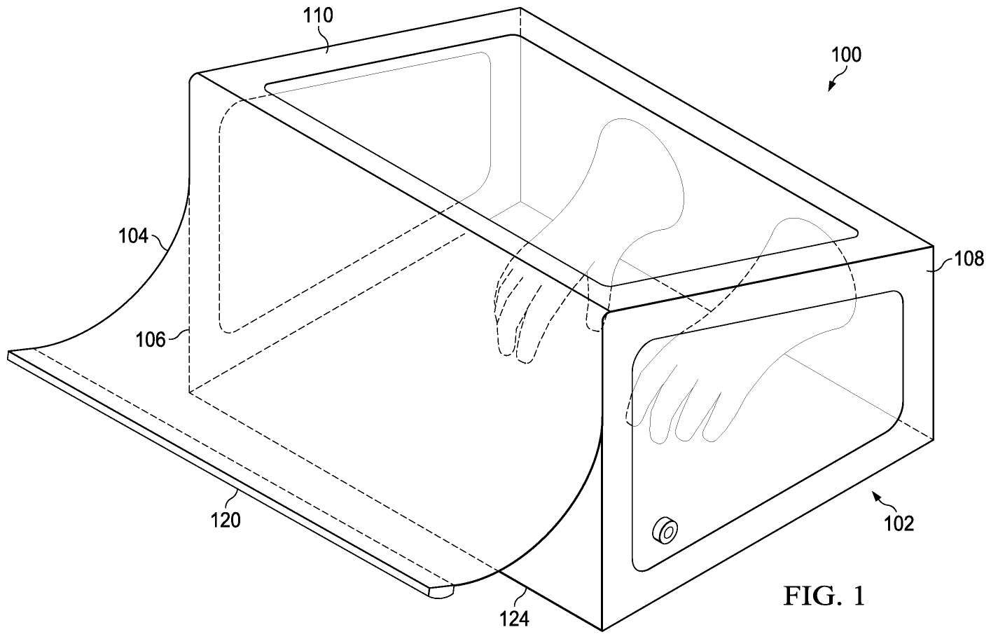

Referring to FIG. 1, isolette 100 will be further described. Isolette 100 includes base frame subassembly 102 and drape subassembly 104.

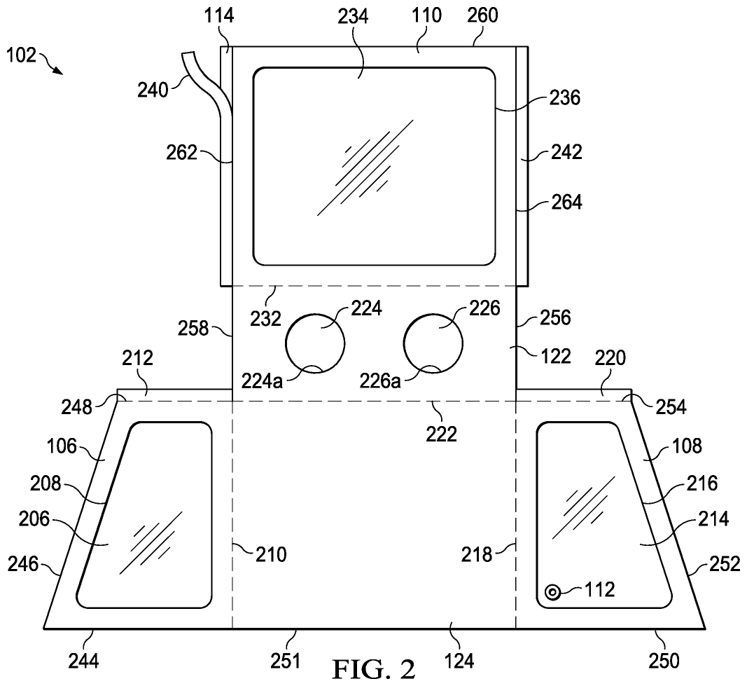

Referring to FIG. 2, base frame subassembly 102 will be further described. Base frame subassembly 102 includes side panel 106, side panel 108, front panel 122, bottom panel 124, and top panel 110. Base frame subassembly 102 is preferably comprised of a semi-flexible flat sheet such as corrugated plasticized cardboard, corriboard or coroplast.

Side panel 106 is bounded by rear edge 244, angled edge 246, and front edge 248. Side panel 106 is connected to bottom panel 124 along fold line 210. Window 206 is affixed to side panel 106 at its perimeter along trapezoidal interface 208. Adhesive strip 212 is positioned along front edge 248.

Side panel 108 is bounded by rear edge 250, angled edge 252, and front edge 254. Side panel 108 is connected to bottom panel 124 along fold line 218. Window 214 is affixed to side panel 108 at its perimeter along trapezoidal interface 216. Window 214 includes pressure port connector 112. Pressure port connector 112 preferably a slip connector or other quick disconnect connector. Adhesive strip 220 is attached along front edge 254.

Front panel 122 is bounded by side edge 256 and side edge 258. Front panel 122 is connected to bottom panel 124 along fold line 222 and to top panel 110 along fold line 232. Front panel 122 includes access ports 224 and 226, as will be further described. In this embodiment, the access ports further comprise removable plugs 224a and 226a, connected to the front panel by micro perforations.

Top panel 110 is bounded by rear edge 260, top edge 262, and top edge 264. Window 234 is affixed to top panel 110 along its perimeter along rectangular interface 236. Adhesive strip 114 is positioned along top edge 262. Adhesive strip 242 is positioned along top edge 264. Adhesive strip 114 includes removable backing 240. In a preferred embodiment, adhesive strips 242, 212, and 220 all include similar removable backings.

Bottom panel 124 is bounded by rear edge 251, fold line 218, fold line 222 and fold line 210. Fold lines 210 and 218 are preferably about 18 inches in length. Fold line 222 and rear edge 251 are preferably about 31 inches in length. Other dimensions will suffice.

Windows 206, 214, and 234 are preferably constructed from a transparent polyethylene sheeting between about 8 and 30 mils thick, sealed at their perimeters to each panel by inductive welding. Other generally transparent semi-flexible materials will suffice.

Preferably, front panel 122 has a height of about 14 inches, along side edges 258 and 256. Rear edges 244 and 250 preferably have a height of about 20 inches. The difference in height imparts a downward sloping angle to top panel 110 of about 18° when the isolette is assembled which aids in viewing the patient through window 234. Other dimensions will suffice.

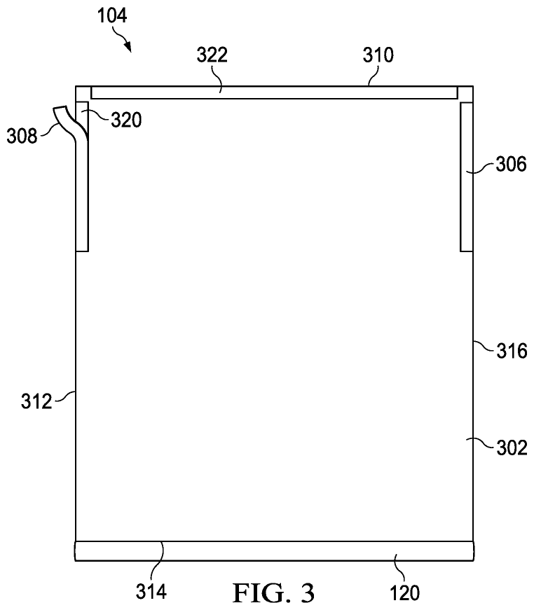

Referring to FIG. 3, drape subassembly 104 will be further described. Drape subassembly 104 includes transparent sheet 302 bonded to flexible seal 120. Transparent sheet 302 is bounded by top edge 310, side 312, bottom edge 314, and side 316. Adhesive strip 322 is affixed along top edge 310, and preferably spans its entire length. Adhesive strip 320 is affixed along side 312. Adhesive strip 306 is affixed along side 316. Adhesive strips 306 and 320 are preferably about 5 inches in length. Adhesive strip 320 includes removable backing 308. Preferably, adhesive strips 306 and 322 include similar removable backings. Flexible seal 120 is affixed along bottom edge 314. Flexible seal 120 is preferably constructed of closed cell butyl rubber foam, about 2 inches in width and ½ inch in height. Transparent sheet 302 is preferably constructed of a clear polyethylene sheeting between about 8 and 10 mils thick.

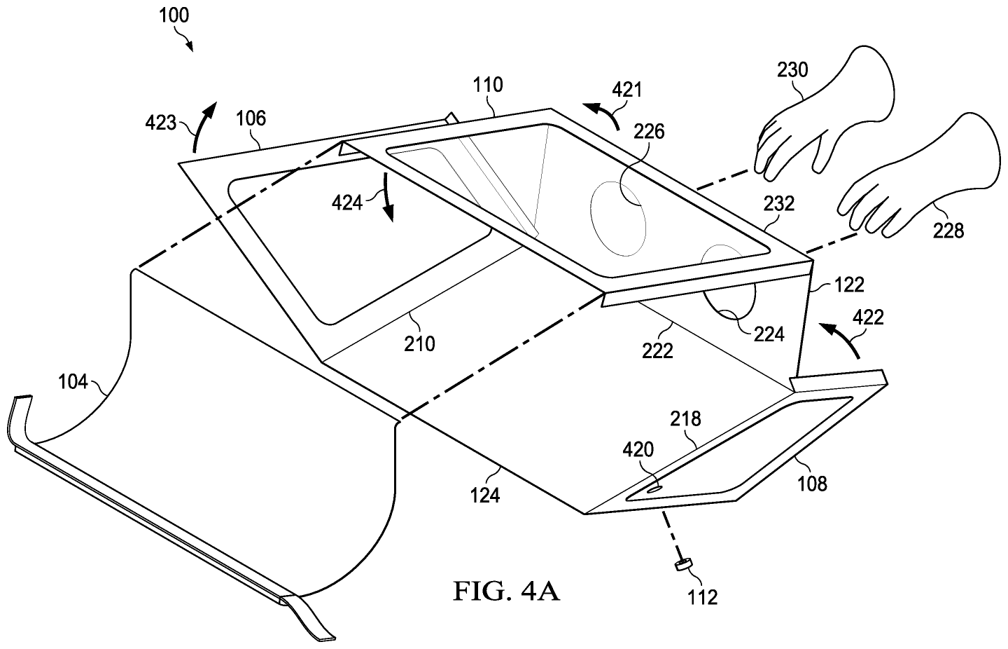

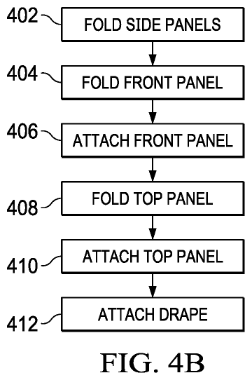

Referring to FIG. 4A, attachments to isolette 100 will be further described. Bi-directional gloves 230 and 228 are attached to the outside face of front panel 122, within access ports 226 and 224. Bi-directional gloves 228 and 230 may be used with either hand and are positioned with the thumb directed upward with respect to the isolette. Pressure port connector 112 is attached to side panel 108 within and sealing hole 420.

Referring to FIGS. 2, 3, 4A and 4B, a method of assembly of isolette 100 will be further described.

At step 402, side panel 106 is folded in direction 423 along fold line 210 until substantially perpendicular to bottom panel 124. Side panel 108 is folded in direction 422 along fold line 218 until substantially perpendicular to bottom panel 124.

At step 404, front panel 122 is folded along fold line 222 in direction 421 until substantially perpendicular to bottom panel 124.

At step 406, front panel 122 is attached to side panels 106 and 108 by adhering adhesive strip 212 to side edge 258 and adhesive strip 220 to side edge 256.

At step 408, top panel 110 is folded in direction 424 along fold line 232 until top panel 110 is adjacent angled edge 246 and angled edge 252.

At step 410, top panel 110 is attached to side panel 106 and side panel 108, by adhering adhesive strip 114 to angled edge 246 and adhesive strip 242 to angled edge 252.

At step 412, drape subassembly 104 is attached to the isolette by adhering adhesive strip 322 along rear edge 260, adhesive strip 320 along rear edge 244 and adhesive strip 306 along rear edge 250.

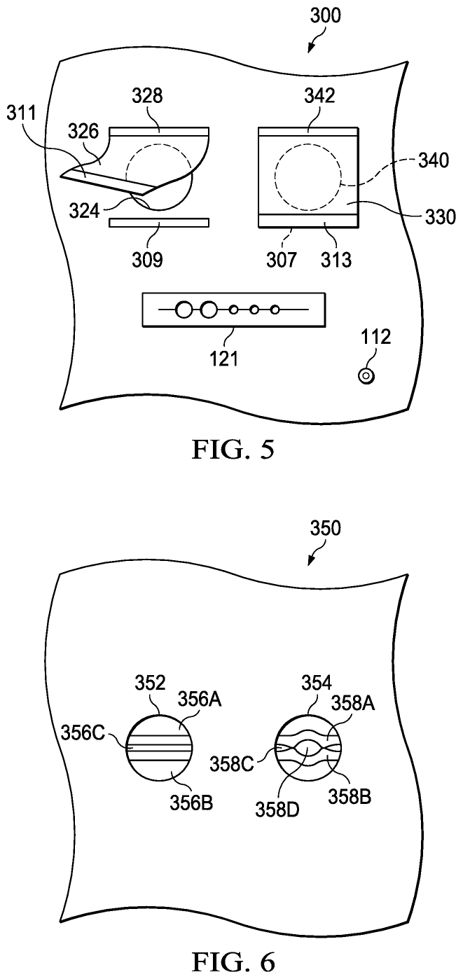

Referring to FIG. 5, an alternate embodiment of a panel, will be further described. All versions of the panels may optionally be employed in all the various embodiments of the isolette.

Panel 300 further comprises access ports 324 and 340. In this example two (2) access ports are provided in a side panel. However, in other embodiments, a single access port in a side panel may be provided. In yet other embodiments, no access ports are provided in a side panel, but is created as needed on an ad hoc basis, as will be further described. Flap 326 is fixed to panel 300 adjacent to and covering access port 324. Flap 326 is fixed to the panel by adhesive 328. Flap 326 further comprises closure 311. Panel 300 further include closure 309. In use, closure 311 is removably affixed to closure 309, thereby sealing flap 326 over access port 324.

Likewise, flap 330 is positioned adjacent to and covering access port 340. Flap 330 is fixed to panel 300 via adhesive 342. Closure 307 is fixed to panel 300. Closure 313 is fixed to flap 330. In use, closure 313 is adapted to removably seal against closure 307, thereby sealing flap 330 over access port 340.

Referring then to FIG. 6, an alternate embodiment of a panel will be further described.

Panel 350 is further comprised of access ports 352 and 354. Access port 352 further comprises iris diaphragms 356a, and 356b. Iris diaphragm 356a overlaps iris diaphragm 356b, in region 356c.

Access port 354 is further comprised of iris diaphragm 358a and 358b. Iris diaphragm 358a overlaps iris diaphragm 358b, in region 358c. Iris diaphragms 358a and 358b, are shown in an open position, accommodating opening 358d.

In a preferred embodiment, the iris diaphragms are comprised of butyl rubber sheet or low-density polyethylene, approximately 10 mils in thickness. The iris diaphragms are fixed to the exterior of the access port, except for the overlap region, which may be parted by physical pressure, to create a ducted portal in the access port, such as opening 358d.

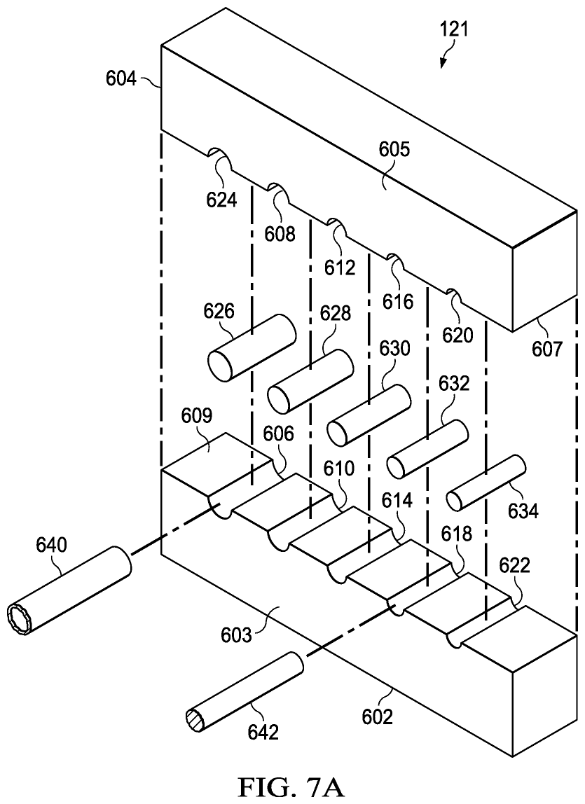

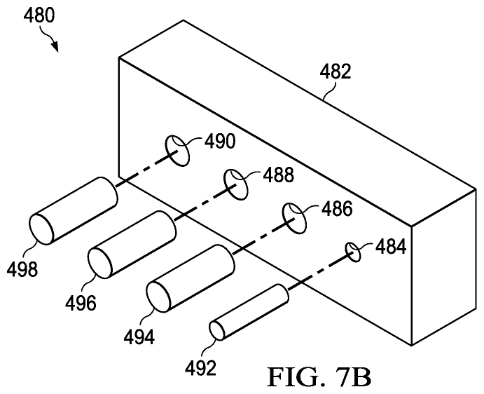

Referring to FIG. 7A, a preferred embodiment of the component access panel will be further described. Component access panel 121 includes top section 604 and bottom section 602. Top section 604 further comprises front face 605 and bottom face 607. Bottom section 602 further comprises front face 603 and top face 609. When assembled, front face 605 and front face 603 are positioned adjacent the inside surface of the side panel and affixed with a suitable medical adhesive. Bottom face 607 is positioned adjacent top face 609.

Semi-cylindrical slots 606, 610, 614, 618, and 622 are formed in the bottom section. Semi-cylindrical slots 624, 608, 612, 616, and 620 are formed in the top section adjacent semi-cylindrical slots 606, 610, 614, 618, and 622, respectively. In general, the slots are all generally parallel and all generally perpendicular to the side panel. When the top and bottom sections are assembled, resealable plug 626 fits within slots 606 and 624. Resealable plug 628 fits within semi-cylindrical slots 608 and 610. Resealable plug 630 fits within semi-cylindrical slots 612 and 614. Resealable plug 632 fits within semi-cylindrical slots 616 and 618. Resealable plug 634 fits within semi-cylindrical slots 620 and 622. The plugs prevent gas leakage through the access panel. In a preferred embodiment, the slots are adapted to seal against the outside surface of different sized tubes and wires, thereby preventing gas leakage through the access panel by insertion of tubes 640 and wires 642, as will be further described. In a preferred embodiment, component access panel 121 is approximately ¼ inch×1 inch cross-section and approximately 8 inches in length and is comprised of closed cell neoprene rubber foam.

Referring to FIG. 7B, an alternate embodiment of the component access panel will be described.

Access panel 480 is comprised of a single integrated block 482. Integrated block 482 includes through holes 484, 486, 488 and 490. Removable plugs 492, 494, 496 and 498 are positioned in through holes 484, 486, 488 and 490. Preferably, the removable plugs are integrally formed with integrated block 482 and held in place by a perforated break line. The block is preferably comprised of closed cell neoprene rubber foam.

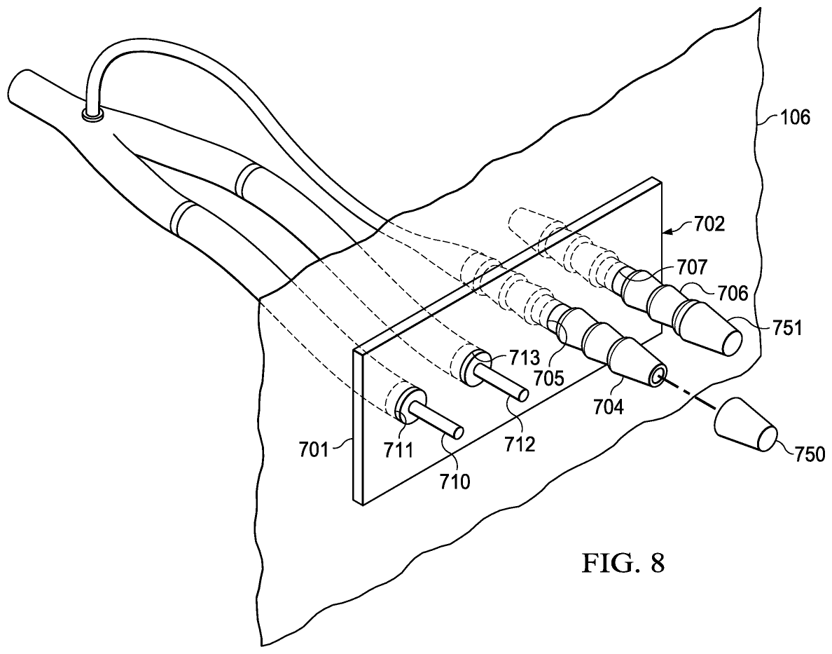

Referring to FIG. 8, an alternate embodiment of the component access panel will be described. Component access panel 702 is generally a semi-rigid plastic connector block supporting several hose connectors. Component access panel 702 further comprises rear surface 701, adhered to side panel 106 with a suitable adhesive.

Component access panel 702 preferably includes a number of double ended connectors forming ducted portals from the exterior of the isolette to its interior. For example, component access panel 702, in this embodiment, includes circuit connector 710, circuit connector 712, circuit connector 704, and circuit connector 706. Circuit connector 710 and circuit connector 712 are adapted to connect to anesthesia lines. Circuit connector 704 and circuit connector 706 are adapted to connect to a source of vacuum. Circuit connector 710 passes through component access panel 702 thru through hole 711. Likewise, circuit connectors 712, 704 and 706 pass through component access panel 702 via through holes 713, 705 and 707, respectively.

In this example, the circuit connectors are adapted to be sealed by caps when not in use, such as cap 750 adapted to mate with circuit connector 704, and cap 751 shown in place on circuit connector 706. In this example, the circuit connectors are either Luer lock type, wedge type, slip type or barb type connectors. Other connector types may be used equally well.

In a preferred embodiment, the component access panel is comprised of a semi flexible polyvinyl chloride sheet, approximately 100 mils thick. The circuit connectors may be adhered in the through holes with a suitable medical grade adhesive sealant.

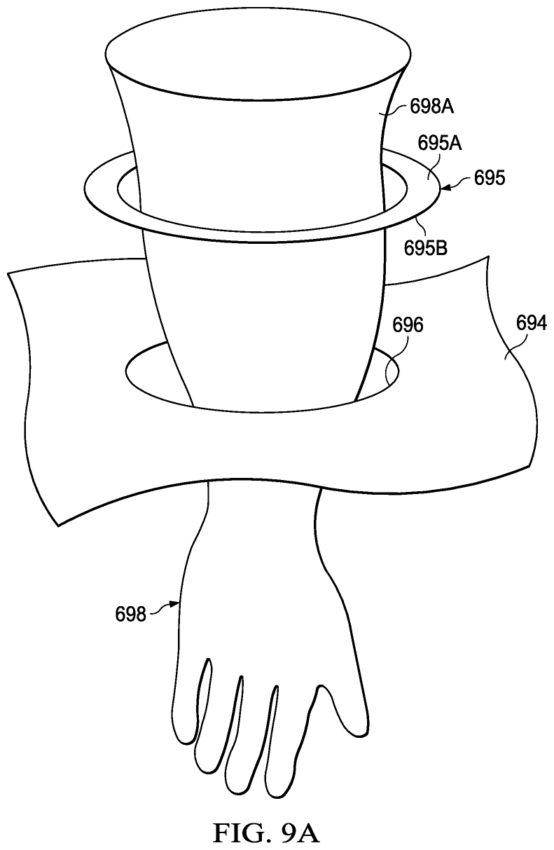

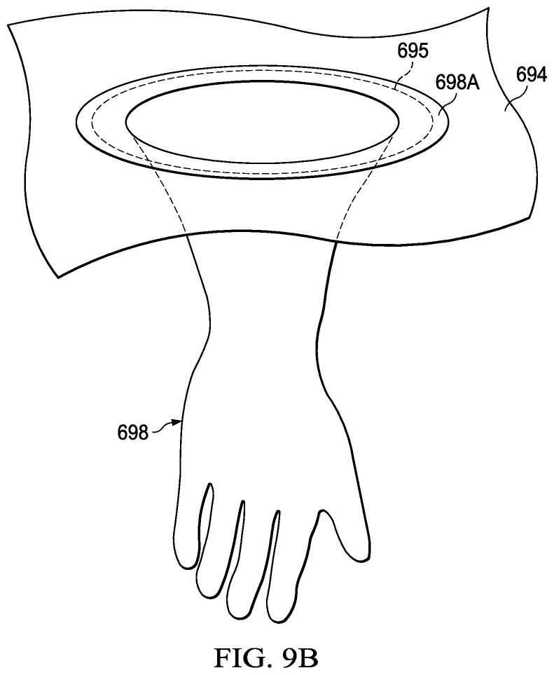

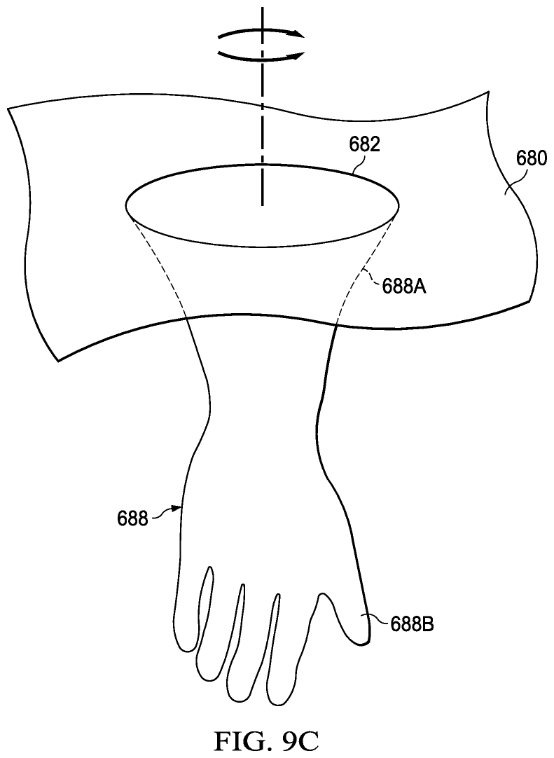

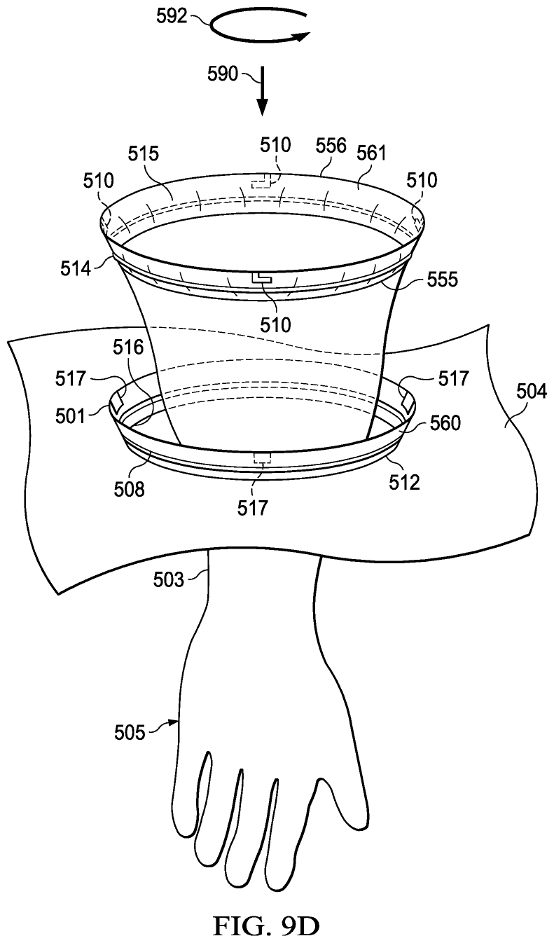

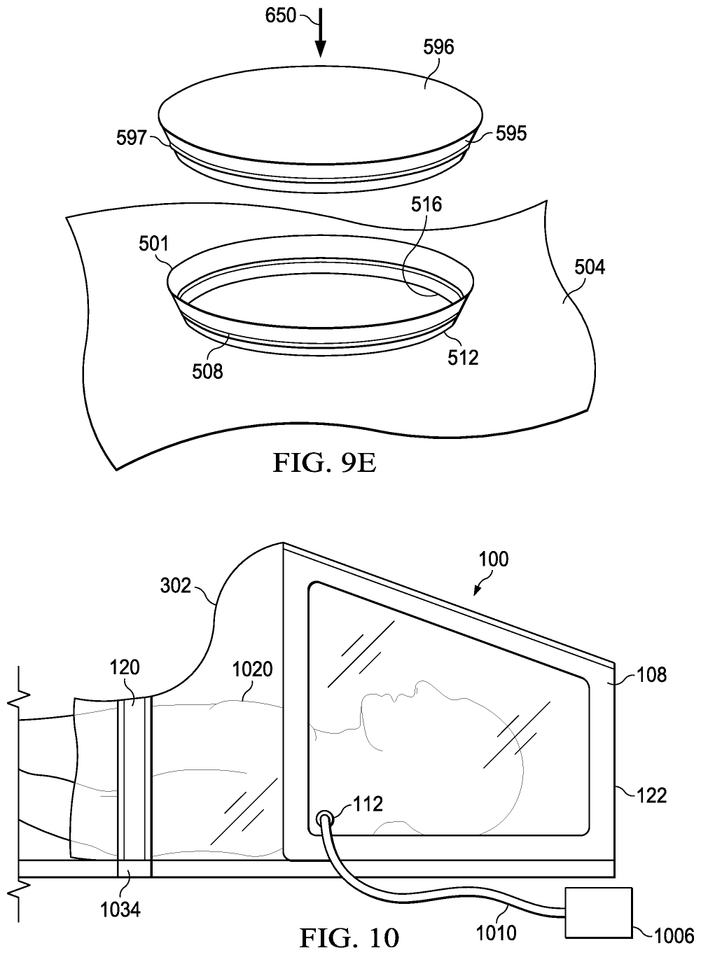

Referring to FIGS. 9A and 9B, an alternate embodiment of an access port will be described.

Panel 694 includes access port 696. Adhesive ring 695 is adapted to circumscribe access port 696. Adhesive ring 695 further comprises adhesive surface 695a and adhesive surface 695b. Glove 698 is adapted to fit within access port 696 and within adhesive ring 695.

Referring then to FIGS. 9A and 9B, when assembled, glove sleeve 698a is fixed to adhesive surface 695a. Panel 694 is fixed to adhesive surface 695b. The connections between glove sleeve 698a, adhesive surface 695a, adhesive surface 695b and panel 694, create an airtight seal, thereby allowing access to the glove from the exterior of the isolette without release of contagions within the isolette.

Referring thing to FIG. 9C, an alternate embodiment of an access port will be described. Panel 680 includes access port 682. Panel 680 is fused to glove sleeve 688a of glove 688, around access port 682, thereby allowing access to the glove from the exterior of the isolette. In this embodiment, glove thumb 688b is oriented vertically up with respect to the isolette, when fused to access port 682. The orientation of the glove is important, because a single “non-handed” glove may be used for either the left, or the right, hand. This embodiment is also important, as will be further described, because it can be placed in any position, in a side, or top panel, on an ad-hoc basis.

Referring to FIG. 9D, an alternate embodiment of an access port will be described. Panel 504 includes connection ring 512 sealed adjacent access port 516 along annular interface 501. The connection ring is sealed at the annular interface with suitable medical adhesive. Connection ring 512 is further comprised of frustroconical body 560. Frustroconical body 560 includes annular O-ring channel 508 on its interior surface. Frustroconical body 560 further includes a plurality of bayonet mount pins 517, dispersed at 90° angles on the interior surface of the frustroconical body relative to a central axis of the frustroconical body.

Glove collar 556 is designed to mate with connection ring 512. Glove collar 556 includes frustroconical body 561. Frustroconical body 561 further includes annular O-ring 514 compressed into exterior annular channel 515. The frustroconical body further includes bayonet mount receptors 510.

Glove 505 is attached to glove collar 556 at sleeve 503 and annular interface 555. In preferred embodiments, sleeve 503 is adhered to annular interface 555 with a suitable medical grade adhesive.

In use, glove 505 is advanced through connection ring 512 and into the interior of the isolette. Glove collar 556 advances in direction 590 into connection ring 512 where annular O-ring 514 seats in O-ring channel 508, thereby forming an air-tight seal. Once the O-ring is seated, glove collar 556 is rotated in direction 592 to lock bayonet mount receptors 510 into position adjacent bayonet mount pins 517.

Referring to FIG. 9E, an alternate embodiment of a connection ring, will be further described. Connection ring 512 includes O-ring channel 508 adjacent its interior annular surface. Barrier cap 596 includes frustroconical body 595 and is generally adapted to seal to the connection ring. Frustroconical body 595 includes annular O-ring 597. O-ring 597 is adapted to seat within O-ring channel 508 as barrier cap 596 is advanced into access port 516 in direction 650. Barrier cap 596 is removably fixed in the access point and creates an airtight seal between the exterior of the isolette and the interior of the isolette.

Referring to FIG. 10, isolette 100 in shown in use. Patient 1020 is positioned inside the isolette on the bottom panel. Transparent sheet 302 is extended from a position above the isolette downward and around the patient. Optional flexible seal 120 is secured around the patient. Optional strap 1034 is secured to hold the flexible seal in place. Negative pressure tube 1010 is shown connected to pressure port connector 112 to provide negative pressure to the isolette from filtered negative pressure turbine 1006.

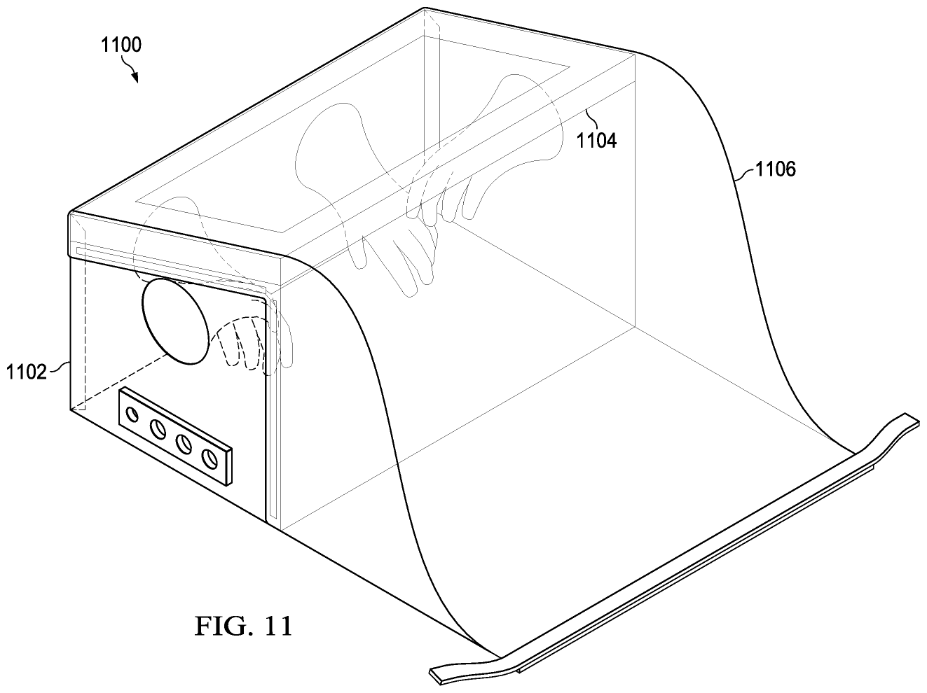

Referring to FIG. 11, isolette 1100 will be further described. Isolette 1100 includes base frame subassembly 1102, top subassembly 1104, and drape subassembly 1106.

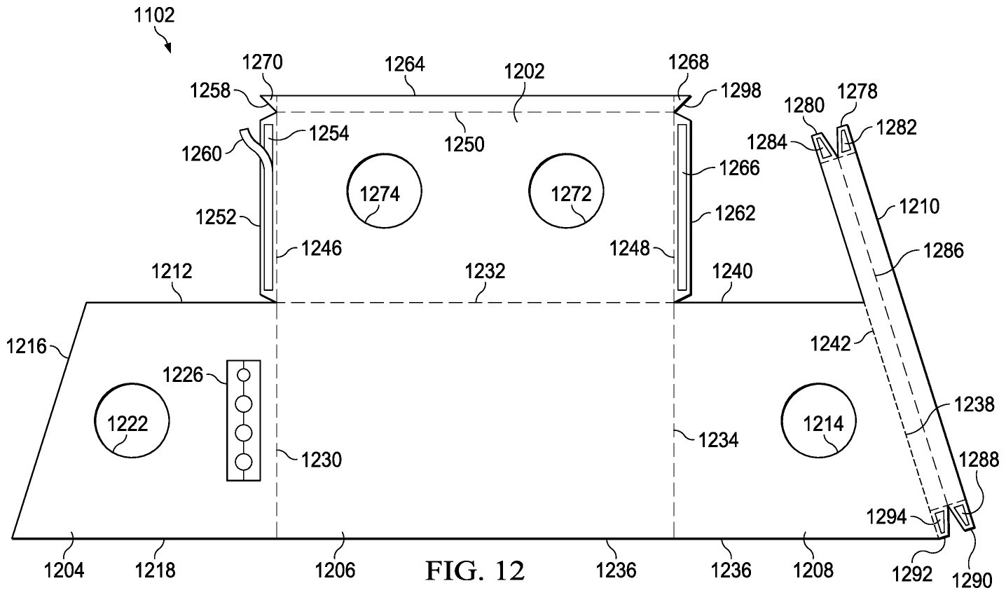

Referring to FIG. 12, base frame subassembly 1102 will be further described. Base frame subassembly 1102 includes front panel 1202, side panel 1204, bottom panel 1206, side panel 1208, and removable cross brace 1210.

Front panel 1202 is bounded by perforated fold line 1232, flap 1252, flap 1264 and flap 1262 and includes access ports 1272 and 1274. In a preferred embodiment, the access ports comprise holes in the sheet material which forms the panels, as will be further described. Front panel 1202 is connected to flap 1264 by perforated fold line 1250. Flap 1264 includes front tab 1298 with adhesive portion 1268 and front tab 1258 with adhesive portion 1270. Front panel 1202 is connected to flap 1262 by perforated fold line 1248. Flap 1262 includes adhesive strip 1266. Front panel 1202 is connected to flap 1252 by perforated fold line 1246. Flap 1252 includes adhesive strip 1254. Removable cover strip 1260 is adhered to adhesive strip 1254 as a removable protective layer. In a preferred embodiment, all the adhesive strips are protected by similar removable cover strips. Front panel 1202 is connected to bottom panel 1206 by perforated fold line 1232.

Side panel 1204 is bounded by front edge 1212, top edge 1216, rear edge 1218 and perforated fold line 1230. Side panel 1204 is connected to bottom panel 1206 by perforated fold line 1230. Side panel 1204 further includes access port 1222 and component access panel 1226, examples of which have been previously described.

Side panel 1208 is bounded by front edge 1240, top edge 1238, rear edge 1236 and perforated fold line 1234. Side panel 1208 is connected to bottom panel 1206 by perforated fold line 1234. Side panel 1208 further includes access port 1214.

In a preferred embodiment, front edges 1240 and 1212 are about 14 inches in length, and rear edges 1236 and 1218 are about 20 inches in length. The bottom panel is preferably about 18 inches deep, along perforated fold lines 1230 and 1234. The difference the lengths of the front and rear edges is important because it imparts a downward slope to the top of the isolette, which aids in viewing the patient when the isolette is in use. The downward slope is about 18°.

Cross brace 1210 is attached to side panel 1208 at perforated tear line 1242. The cross brace is removable along the tear line and is used as a structural stiffener in the final assembly, as will be further described. Tabs 1278, 1280, 1290, and 1292 are attached to cross brace 1210 and are used to secure the cross brace in place, as will be further described. Tab 1278 includes adhesive portion 1282. Tab 1280 includes adhesive portion 1284. Tab 1290 includes adhesive portion 1288. Tab 1292 includes adhesive portion 1294.

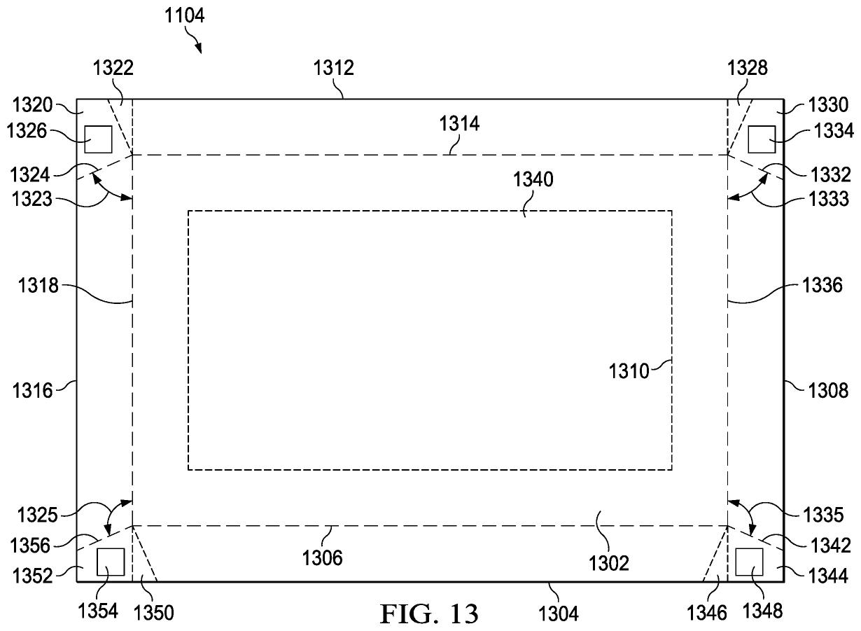

Referring to FIG. 13, top subassembly 1104 will be further described. Top subassembly 1104 includes top panel 1302. Top panel 1302 includes removable window plug 1340 bounded at its perimeter along perforated tear line 1310.

Top panel 1302 is bounded by flap 1304, flap 1308, flap 1312 and flap 1316.

Flap 1304 is connected to top panel 1302 along perforated fold line 1306.

Flap 1316 is connected to top panel 1302 along perforated fold line 1318. Flap 1316 is connected to corner tab 1352 along perforated fold line 1356. Perforated fold line 1356 makes angle 1325 with perforated fold line 1318. In a preferred embodiment, angle 1325 is about 108°. Flap 1316 is connected to corner tab 1320 along perforated fold line 1324. Perforated fold line 1324 makes angle 1323 with perforated fold line 1318. In a preferred embodiment, angle 1323, is about 72°. Corner tab 1352 includes adhesive strip 1354. Corner tab 1320 includes adhesive strip 1326.

Flap 1312 is connected to top panel 1302 along perforated fold line 1314.

Flap 1308 is connected to top panel 1302 along perforated fold line 1336. Flap 1308 is connected to corner tab 1330 along perforated fold line 1332. Perforated fold line 1332 makes angle 1333 with perforated fold line 1336. In an alternate embodiment, angle 1333 is about 72°. Flap 1308 is connected to corner tab 1344 along perforated fold line 1342. Perforated fold line 1342 makes angle 1335 with perforated fold line 1336. In a preferred embodiment, angle 1335 is about 108°.

Angles 1323, 1325, 1333 and 1335 are important because they enable flap 1312 and flap 1304 to be positioned fully in contact with front panel 1202 and rear edges 1218 and 1236, respectively.

Removable plug 1350 is located between flap 1304 and corner tab 1352. Removable plug 1322 is located between corner tab 1320 and flap 1312. Removable plug 1328 is located between flap 1312 and corner tab 1330. Removable plug 1346 is located between flap 1304 and corner tab 1344.

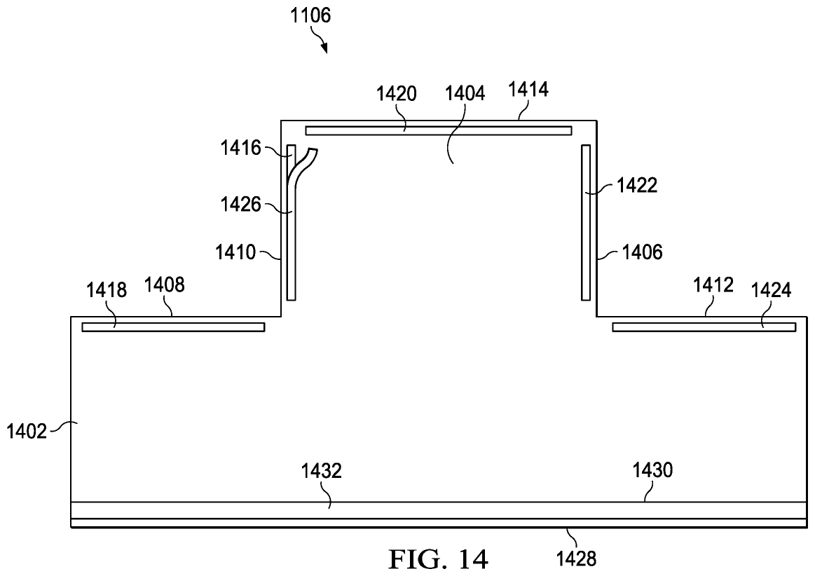

Referring to FIG. 14, drape subassembly 1106 will be further described. Drape subassembly 1106 includes lower portion 1402 and upper portion 1404. Lower portion includes back edge 1428, front edge 1408 and front edge 1412. Flexible seal 1432 is adhered to drape subassembly 1106 along back edge 1428 at adhesive interface 1430. Flexible seal 1432 is preferably constructed of closed cell butyl rubber foam stripping. Lower portion 1402 further includes adhesive strip 1418 and adhesive strip 1424. Adhesive strip 1418 is adhered to the lower portion adjacent front edge 1408. Adhesive strip 1424 is adhered to the lower portion adjacent front edge 1412.

Upper portion 1404 includes side edge 1410, front edge 1414, and side edge 1406. Adhesive strip 1416 is adhered to upper portion 1404 adjacent side edge 1410. Adhesive strip 1416 includes cover strip 1426. Cover strip 1426 is removed from adhesive strip 1416 during assembly, as will be further described. Adhesive strip 1420 is adhered to upper portion 1404 adjacent front edge 1414. Adhesive strip 1422 is adhered to upper portion 1404 adjacent side edge 1406. Preferably adhesive strips 1418, 1420, 1422, and 1424 further include cover strips similar to cover strip 1426.

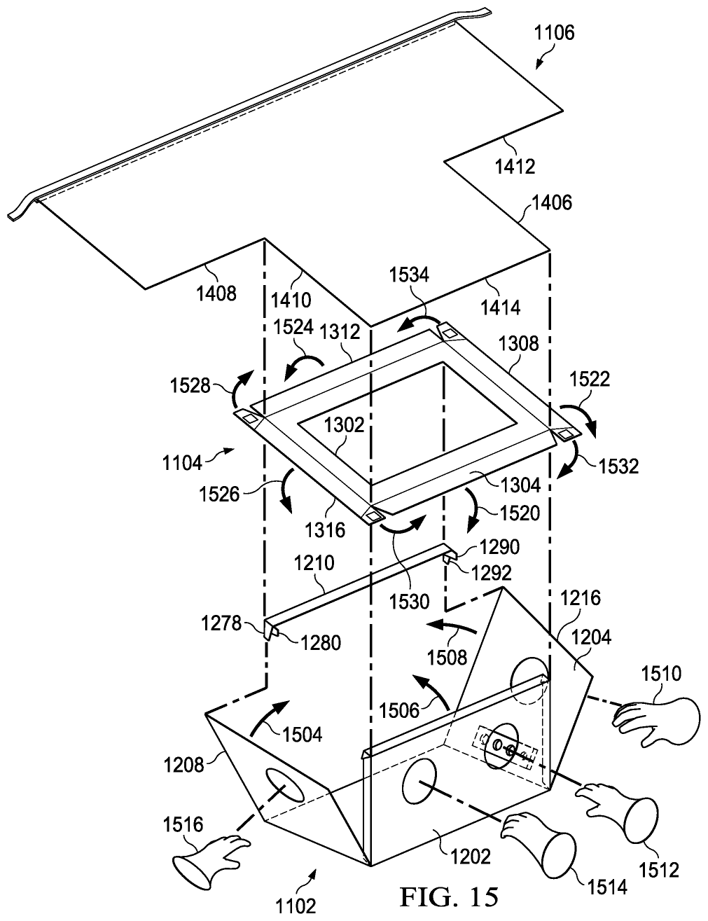

Referring to FIGS. 12 and 15, the base frame assembly will be further described. Glove 1510 is adhered to side panel 1204 in access port 1222. Glove 1512 is adhered to front panel 1202 in access port 1274. Glove 1514 is adhered to front panel 1202 in access port 1272. Glove 1516 is adhered to side panel 1208 in access port 1214. Preferably, gloves 1510, 1512, 1514, and 1516 are fixed and sealed to the side panel, in a thumbs-up position, with a suitable medical grade adhesive.

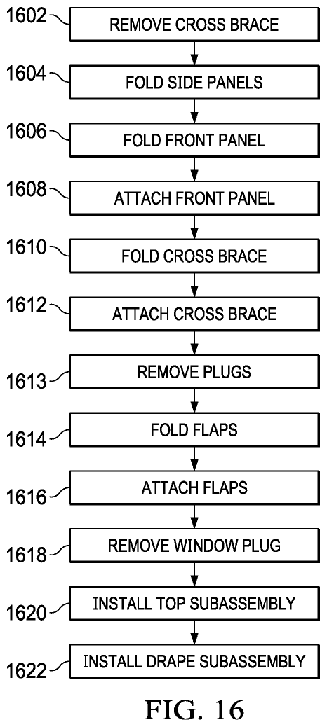

Referring to FIGS. 12, 13, 14, 15 and 16, a method of assembly of isolette 1100 will be further described.

At step 1602, cross brace 1210 is removed from side panel 1208 by separating it along perforated tear line 1242.

At step 1604, side panel 1204 is folded along perforated fold line 1230 in direction 1508 until substantially perpendicular to bottom panel 1206. Side panel 1208 is folded along perforated fold line 1234 in direction 1504 until substantially perpendicular to bottom panel 1206.

At step 1606, front panel 1202 is folded along perforated fold line 1232 in direction 1506 until substantially perpendicular to bottom panel 1206. Flap 1252 is folded toward side panel 1204 along perforated fold line 1246. Flap 1264 is folded toward front panel 1202 along perforated fold line 1250. Flap 1262 is folded toward side panel 1208 along perforated fold line 1248.

At step 1608, the cover strips are removed from adhesive portions 1270 and 1268 and adhesive strips 1254 and 1266. Adhesive strip 1254 is adhered to front edge 1212. Adhesive portion 1270 is adhered to top edge 1216. Adhesive strip 1266 is adhered to front edge 1240. Adhesive portion 1268 is adhered to top edge 1238.

At step 1610, cross brace 1210 is folded longitudinally along perforated fold line 1286.

At step 1612, cross brace 1210 is attached to side panel 1204 by adhesive portions 1284 and 1282, to side panel 1208 by adhesive portions 1294 and 1288, and to top edge 1238 by adhesive portion 1282. Cross brace 1210 provides stability to base frame subassembly 1102.

At step 1613, construction of the top subassembly begins by removing and discarding plugs 1350, 1346, 1328 and 1322.

At step 1614, flap 1308 is folded along perforated fold line 1336 in direction 1522 until flap 1308 is substantially perpendicular to top panel 1302. Flap 1316 is folded along perforated fold line 1318 in direction 1526 until flap 1316 is substantially perpendicular to top panel 1302. Flap 1304 is folded along perforated fold line 1306 in direction 1520 until flap 1304 is adjacent fold lines 1356 and 1342. Flap 1312 is folded along perforated fold line 1314 in direction 1524 until flap 1312 is adjacent fold lines 1324 and 1332. Tab 1352 is folded along fold line 1356 in direction 1530. Tab 1320 is folded along fold line 1324 in direction 1528. Tab 1344 is folded along fold line 1342 in direction 1532. Tab 1330 is folded along fold line 1332 in direction 1534.

At step 1616, adhesive strips 1354 and 1348 are adhered to flap 1304. Adhesive strips 1334 and 1326 are adhered to flap 1312.

At step 1618, window plug 1340 is removed from top panel 1302 along perforated tear line 1310.

At step 1620, top subassembly 1104 is installed on base frame subassembly 1102. Flap 1304 is positioned adjacent to front panel 1202. Flap 1308 is positioned adjacent side panel 1204. Flap 1312 is positioned adjacent cross brace 1210. Flap 1316 is positioned adjacent side panel 1208.

At step 1622, drape subassembly 1106 is installed. The protective covers from all adhesive strips are removed. Adhesive strips 1416, 1420, and 1422 are adhered to top panel 1302 around its outside perimeter. Adhesive strip 1418 is adhered to side panel 1208. Adhesive strip 1424 is adhered to side panel 1204.

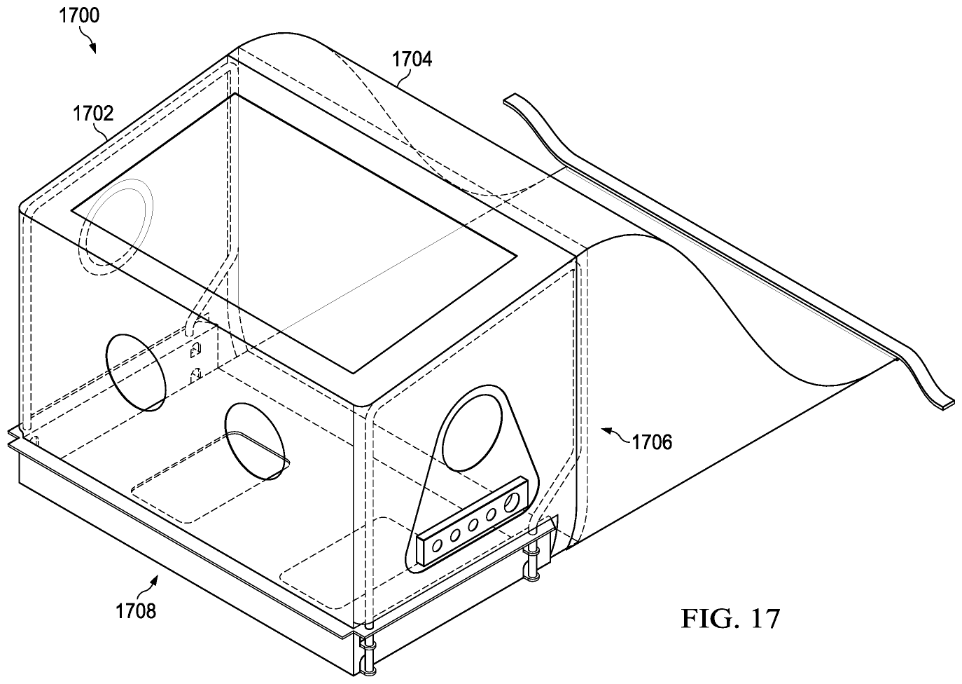

Referring to FIG. 17, isolette 1700 will be further described. Isolette 1700 includes base subassembly 1708, support frame subassembly 1706, enclosure subassembly 1702, and drape subassembly 1704.

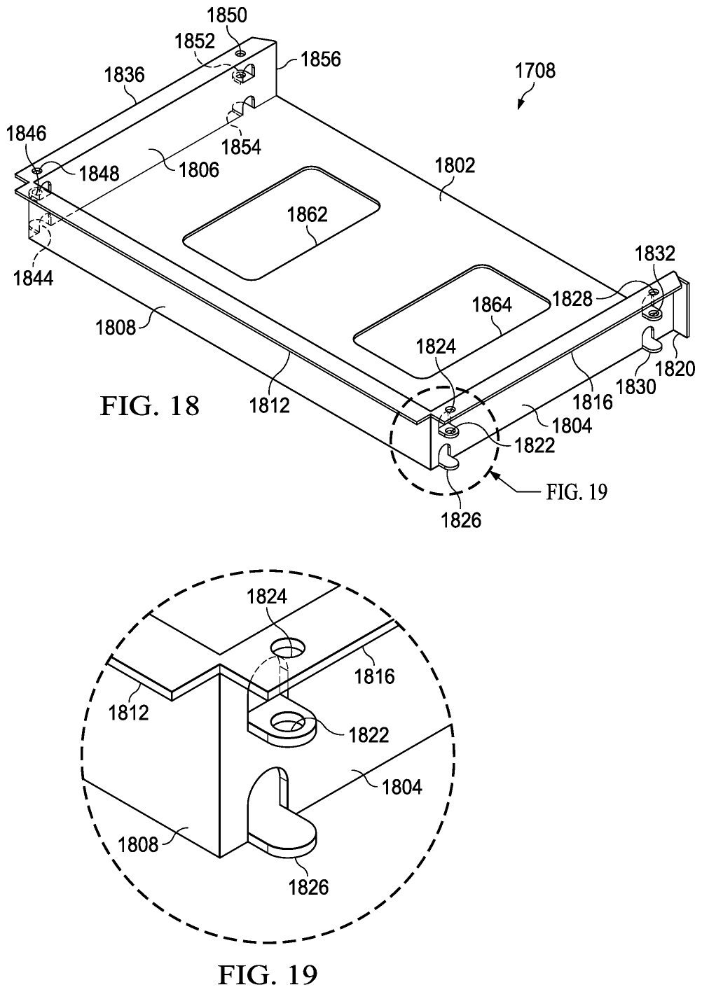

Referring to FIGS. 18 and 19, base subassembly 1708 will be further described. Base subassembly 1708 includes base panel 1802, right wall 1804, left wall 1806, and front wall 1808. Right wall 1804, left wall 1806 and front wall 1808 are substantially perpendicular to base panel 1802. In a preferred embodiment, base panel 1802, right wall 1804, left wall 1806 and front wall 1808 are integrally formed. Base panel 1802 further includes openings 1862 and 1864 in order to reduce weight. Anchor tab 1830, anchor tab 1826, anchor tab 1844 and anchor tab 1854 extend from and are substantially coplanar with base panel 1802, as will be further described.

Right wall 1804 includes back flange 1820, horizontal flange 1816, eyelet 1832 and eyelet 1822. Back flange 1820 is substantially perpendicular to right wall 1804 and base panel 1802. Horizontal flange 1816 is substantially perpendicular to right wall 1804 and substantially parallel to base panel 1802. Horizontal flange 1816 includes anchor holes 1828 and 1824, positioned directly above and coaxial with eyelets 1832 and 1822, respectively.

Left wall 1806 includes back flange 1856, horizontal flange 1836, eyelet 1852 and eyelet 1846. Back flange 1856 is substantially perpendicular to left wall 1806 and substantially parallel to base panel 1802. Horizontal flange 1836 is substantially perpendicular to left wall 1806 and substantially parallel to base panel 1802. Horizontal flange 1836 includes anchor hole 1848 and anchor hole 1850, positioned directly above and coaxial with eyelets 1846 and 1852, respectively.

Referring to FIG. 19, base subassembly 1708 will be further described. Anchor tab 1826 extends perpendicularly from right wall 1804. Eyelet 1822 extends perpendicularly from right wall 1804. Anchor hole 1824 is positioned in horizontal flange 1816 directly above and coaxial with eyelet 1822. Anchor hole 1824, eyelet 1822 and anchor tab 1826 are vertically aligned and create a 3-point support arrangement for anchor pin 2032, as will be further described. A similar 3-point support arrangement is created by anchor hole 1828, eyelet 1832 and anchor tab 1830 on right wall 1804, and anchor hole 1848, eyelet 1846 and anchor tab 1844 and anchor hole 1850, eyelet 1852, and anchor tab 1854 on left wall 1806.

Base subassembly 1708 is preferably constructed from 20-gauge stainless steel or aluminum plate stock. In a preferred embodiment, base assembly 1708 is formed from a single integrated sheet. In another preferred embodiment, the side walls and flanges are formed separately and welded to the base panel. The metallic material allows for base subassembly 1708 to be repeatedly autoclaved and sterilized for reuse, as will be further described.

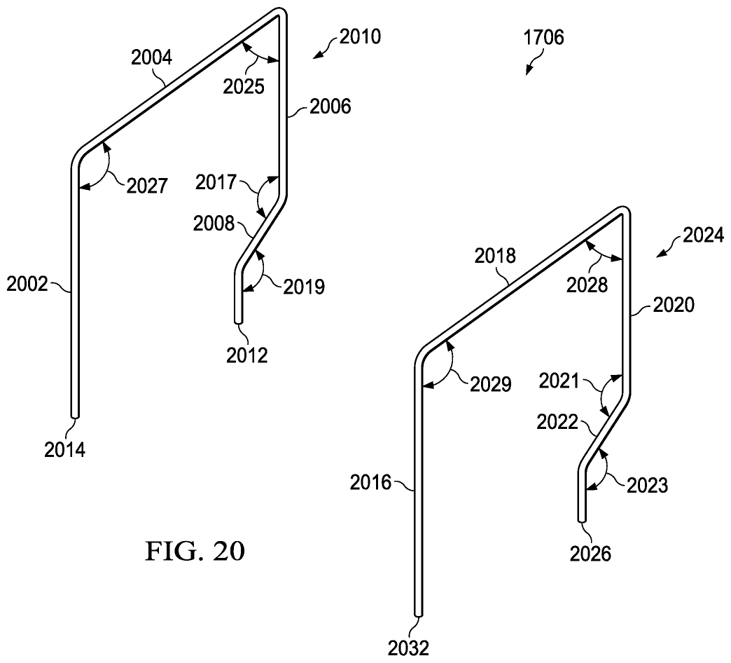

Referring to FIG. 20, support frame subassembly 1706 will be further described. Support frame subassembly 1706 includes right support frame 2024 and left support frame 2010.

Right support frame 2024 includes front stanchion 2016 attached to anchor pin 2032 and top rail 2018 at angle 2029 of about 97°. Top rail 2018 is attached to rear stanchion 2020 at angle 2028 of about 83°. Rear stanchion 2020 is attached to angled segment 2022 at angle 2021 of about 45°. Angled segment 2022 is attached to anchor pin 2026 at angle 2023 of about 45°. In a preferred embodiment anchor pin 2032, front stanchion 2016, top rail 2018, rear stanchion 2020, angled segment 2022, and anchor pin 2026 are integrally formed from a stainless steel or fiberglass rod stock of about a ¼ inch diameter.

Left support frame 2010 includes front stanchion 2002 attached to anchor pin 2014 and top rail 2004 at angle 2027 of about 97°. Top rail 2004 is attached to rear stanchion 2006 at angle 2025 of about 83°. Rear stanchion 2006 is attached to angled segment 2008 at angle 2017 of about 45°. Angled segment 2008 is attached to anchor pin 2012 at angle 2019 of about 45°. In a preferred embodiment anchor pin 2014, front stanchion 2002, top rail 2004, rear stanchion 2006, angled segment 2008 and anchor pin 2012 are integrally formed from stainless steel or fiberglass rod stock of about a ¼ inch diameter.

In a preferred embodiment, right support frame 2024 and left support frame 2010 are mirror images. Angles 2028, 2029, 2027 and 2025 are important because they support the top subassembly at a downward sloping angle toward the front of the isolette to aid in viewing the patient.

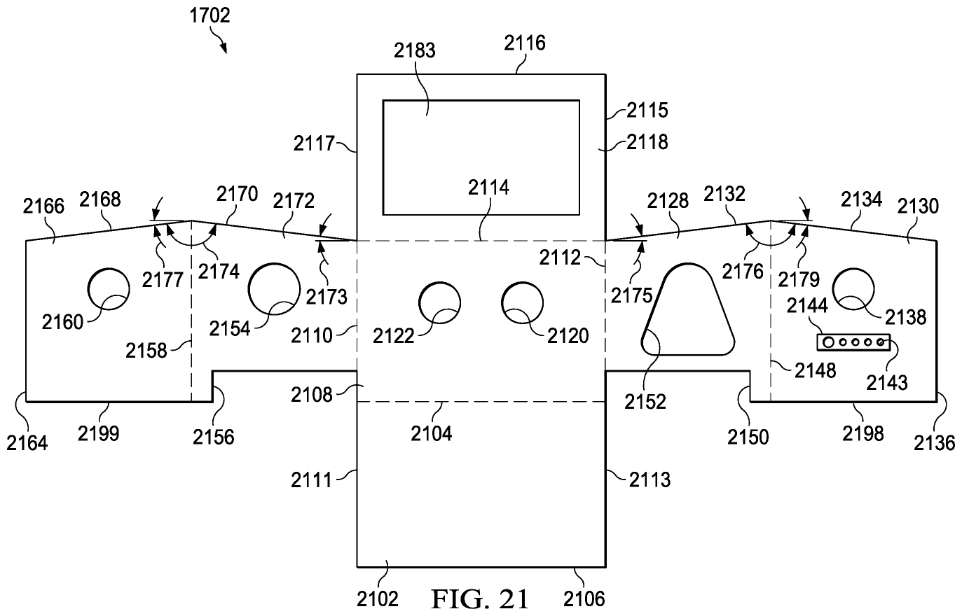

Referring to FIG. 21, enclosure subassembly 1702 will be further described. Enclosure subassembly 1702 includes top sheet 2118, front sheet 2108, bottom sheet 2102, left interior sheet 2172, left exterior sheet 2166, right interior sheet 2128, and right exterior sheet 2130.

Top sheet 2118 is bounded by rear edge 2116, side edge 2115, side edge 2117 and fold line 2114. Fold line 2114 connects top sheet 2118 and front sheet 2108. In a preferred embodiment, rear edge 2116 and fold line 2114 are about 24 inches in length. Preferably, side edges 2115 and 2117 are about 16 inches in length. Top sheet 2118 optionally may include integrally formed transparent window 2183. In a preferred embodiment, the window is constructed of a flexible transparent polyethylene sheet about 20 mils thick, inductively welded to the top sheet around its perimeter.

Bottom sheet 2102 is bounded by rear edge 2106, side edge 2111, side edge 2113 and fold line 2104. Preferably, rear edge 2106 and fold line 2104 are about 24 inches in length. Preferably, side edges 2111 and 2113 are about 16 inches in length. Fold line 2104 connects bottom sheet 2102 and front sheet 2108.

Front sheet 2108 is bounded by fold line 2114, fold line 2112, fold line 2104, and fold line 2110. Preferably, fold lines 2110 and 2112 are about 16 inches in length. Front sheet 2108 further includes access ports 2120 and 2122. The access ports are holes in the sheet material, that can accommodate various closures and gloves, as has be previously described.

Right interior sheet 2128 is bounded by interior angled edge 2132, fold line 2148, base connection bay 2150 and fold line 2112. Right interior sheet 2128 is connected to right exterior sheet 2130 along fold line 2148. Right interior sheet 2128 is connected to front sheet 2108 along fold line 2112. Right interior sheet 2128 further includes interior hole 2152. Interior hole 2152 is generally trapezoidal in shape, to accommodate access panel 2144 and access port 2138, as will be further described. Fold line 2148 is preferably about 18 inches in length. Interior angled edge 2132 is preferably about 16.2 inches in length.

Right exterior sheet 2130 is bounded by exterior angled edge 2134, edge 2136, bottom edge 2198 and fold line 2148. Right exterior sheet 2130 includes access port 2138 and access panel 2144. Access panel 2144 is adhered to right exterior sheet 2130, as previously described. Access panel 2144 further includes pressure port 2143. Preferably, edge 2136 is about 16 inches in length.

Left interior sheet 2172 is bounded by interior angled edge 2170, base connection bay 2156, fold line 2110 and fold line 2158. Left interior sheet 2172 is connected to left exterior sheet 2166 along fold line 2158. Left interior sheet 2172 is connected to front sheet 2108 by fold line 2110. Left interior sheet 2172 further includes interior hole 2154. Interior hole 2154 is generally circular and sized to accommodate access port 2160. Fold line 2158 is preferably about 18 inches in length. Interior angled edge 2170 is about 16.2 inches in length.

Left exterior sheet 2166 is bounded by exterior angled edge 2168, edge 2164 and bottom edge 2199. Left exterior sheet 2166 includes access port 2160. Preferably, edge 2164 is about 16 inches in length.

Exterior angled edge 2168 and interior angled edge 2170 form angle 2174. Angle 2174 is preferably about 166°. Interior angled edge 2132 and exterior angled edge 2134 form angle 2176. In a preferred embodiment, angle 2176 is about 166°.

Angles 2173, 2175, 2177 and 2179 are all about 7°. The angles are important because they accommodate the support frames and impart a downward slope of about 7° to the top sheet when the isolette is assembled. In various embodiments the downward slope aids in viewing the patient from the front of the isolette.

In a preferred embodiment, enclosure subassembly 1702 is constructed from corrugated polyethylene sheeting, such as coroplast or corriboard, between 8 and 30 mils thick. In another embodiment, the enclosure subassembly is constructed of a transparent PVC or marine vinyl sheet stock about 40 mils in thickness. Other thickness may be used so long as the sheeting is flexible and will maintain a crease line.

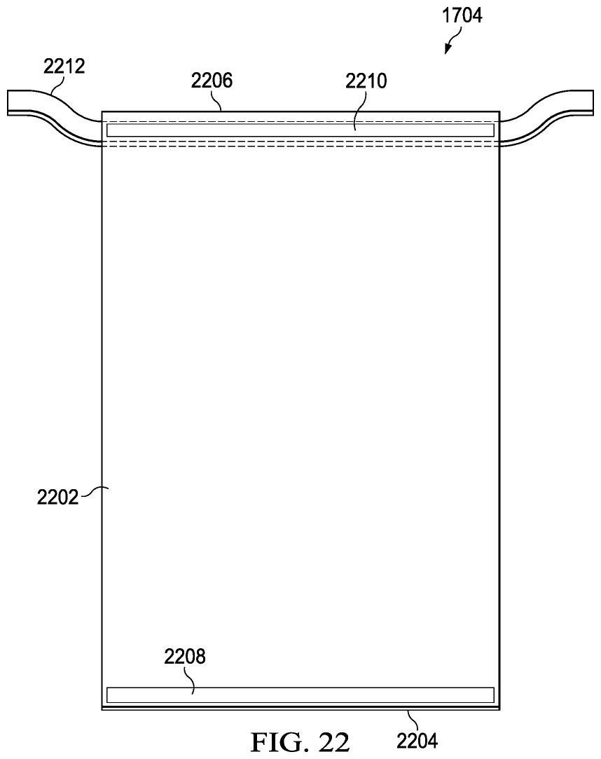

Referring to FIG. 22, drape subassembly 1704 will be further described. Drape subassembly 1704 includes drape 2202. Drape 2202 includes edge 2204, edge 2206, adhesive strip 2208, flexible seal ring 2210 and strap 2212. Adhesive strip 2208 is positioned on drape 2202 adjacent edge 2204. Flexible seal ring 2210 is positioned adjacent to and generally parallel with edge 2206. Strap 2212 is adhered to the drape along edge 2206 and adjacent to flexible seal ring 2210. In a preferred embodiment, strap 2212 is constructed of polyester or polypropylene woven medical strapping about 2 inches wide, and includes terminal Velcro® type closures. In another preferred embodiment, strap 2212 may be medical adhesive tape. Flexible seal ring 2210 is preferably constructed from a closed cell butyl rubber foam strip about ½ inches in thickness and about 2 inches wide.

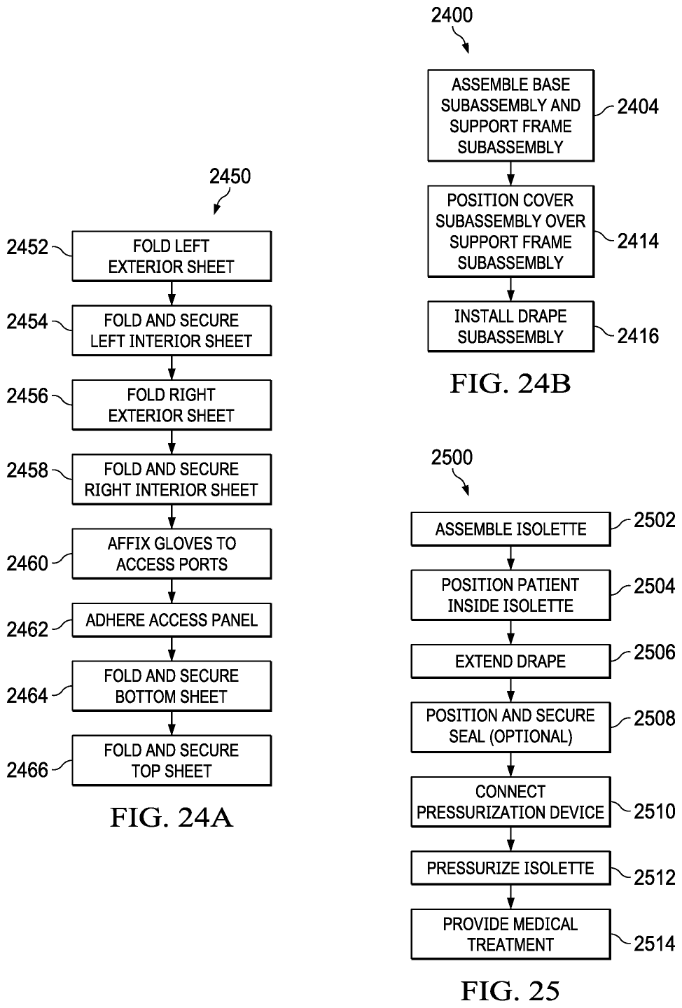

Referring to FIGS. 21, 23A, 23B, 23C, 23D, 23E, 23F and 24A preferred method 2450 of assembly of the isolette, will be further described.

At step 2452, left exterior sheet 2166 is folded in direction 2330, along fold line 2158, until edge 2164 is adjacent front sheet 2108. Adhesive strip 2146 is adhered to front sheet 2108.

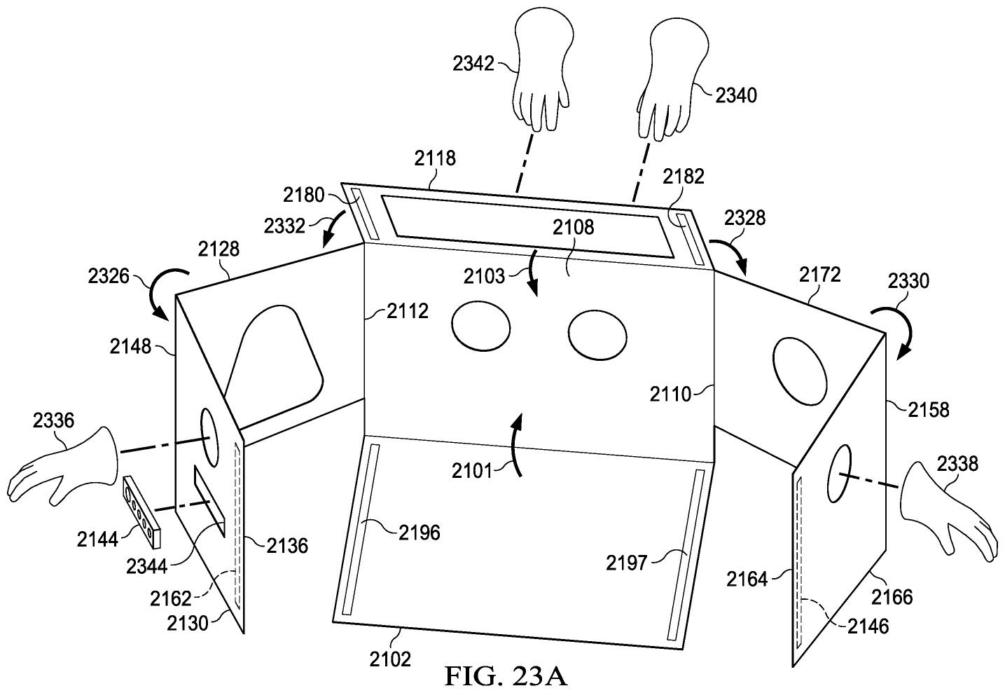

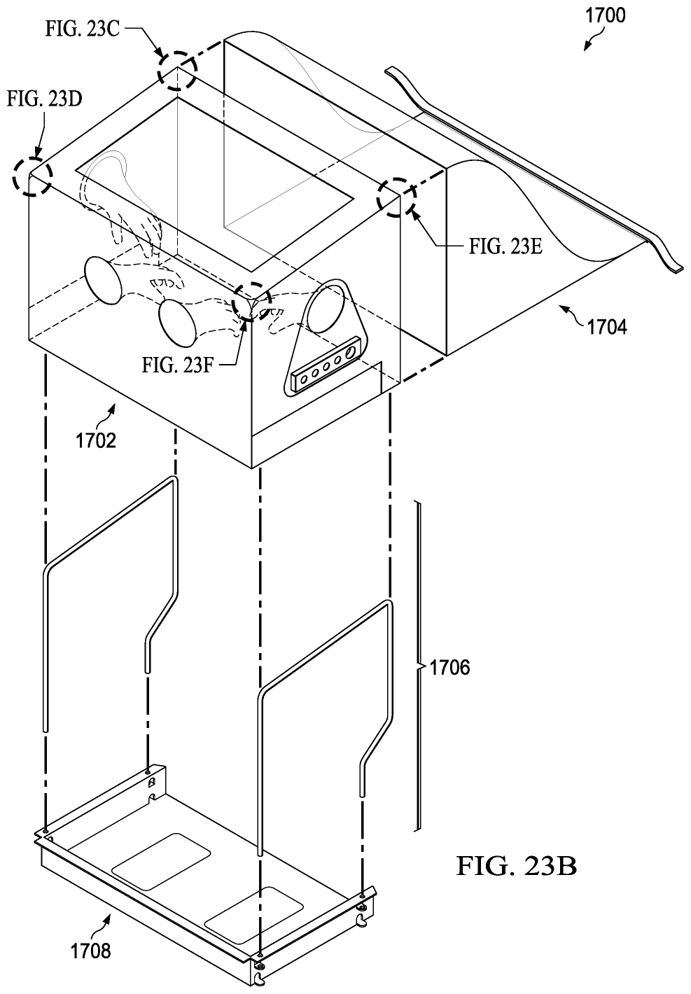

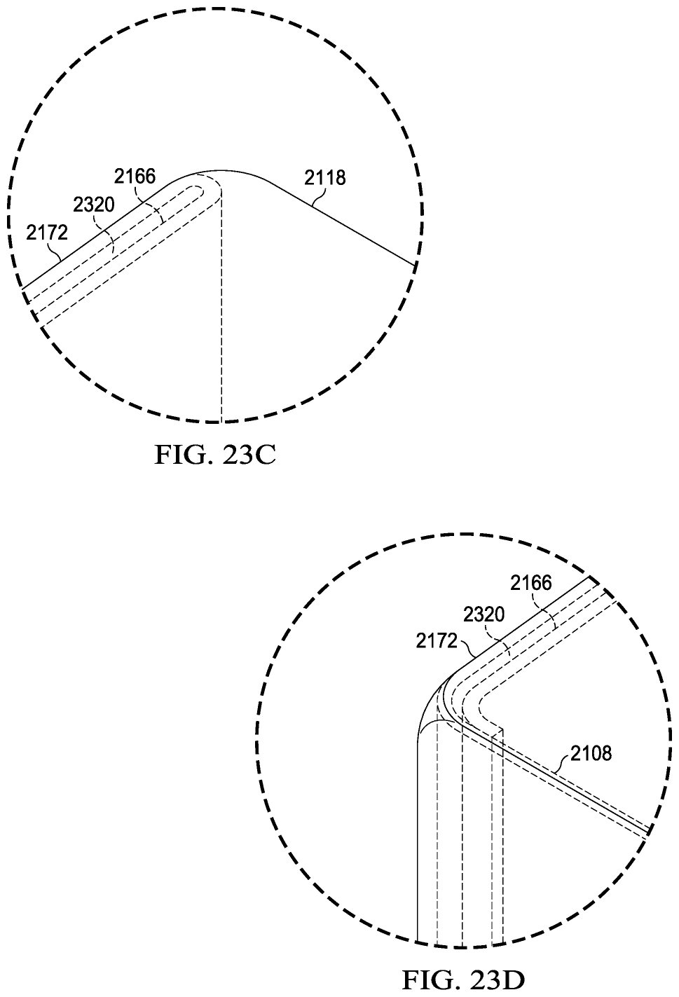

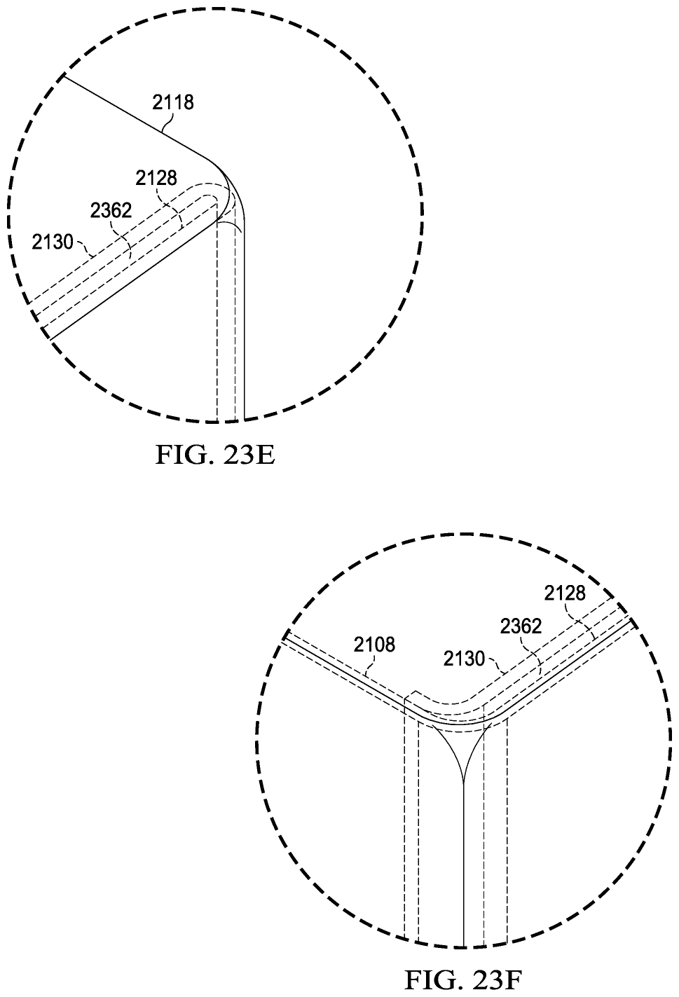

At step 2454, left interior sheet 2172 is folded in direction 2330, along fold line 2158, until left exterior sheet 2166 creates slot 2320 with left interior sheet 2172. The resulting composite sheet is then folded in direction 2328 along fold line 2110 until left interior sheet 2172 is substantially perpendicular to front sheet 2108. As shown in FIG. 23C and FIG. 23D, slot 2320 is created between left interior sheet 2172 and left exterior sheet 2166. Slot 2320 is preferably about ¼ inch in width.

At step 2456, right exterior sheet 2130 is folded in direction 2326, along fold line 2148, until edge 2136 is adjacent front sheet 2108. Adhesive 2162 is adhered to front sheet 2108.

At step 2458, right interior sheet 2128 is folded in direction 2332, along fold line 2112, until right interior sheet 2128 is substantially perpendicular to front sheet 2108. As shown in FIG. 23E and FIG. 23F, slot 2362 is created between right interior sheet 2128 and right exterior sheet 2130.

At step 2460, the gloves are fixed within the access ports, as previously described. In one preferred embodiment, glove 2338 is adhered to left exterior sheet 2166 in access port 2160. Glove 2336 is adhered to right exterior sheet 2130 in access port 2138. Glove 2342 is adhered to front sheet 2108 through access port 2120. Glove 2340 is adhered to front sheet 2108 through access port 2122. The gloves are adhered with a suitable, quick dry rubber cement adhesive in a “thumbs-up” orientation.

At step 2462, access panel 2144 is adhered to right exterior sheet 2130 in hole 2344.

At step 2464, bottom sheet 2102 is folded at fold line 2104, in direction 2101 until bottom sheet 2102 is adjacent bottom edge 2198 and bottom edge 2199 and is substantially perpendicular to front sheet 2108. Bottom sheet 2102 is held in place adjacent bottom edges 2198 and 2199 by adhesive strips 2196 and 2197, respectively.

At step 2466, top sheet 2118 is folded at fold line 2114 in direction 2103 until top sheet 2118 is adjacent interior angled edge 2132 and interior angled edge 2170. Top sheet 2118 is attached to interior angled edge 2132 and interior angled edge 2170 by adhesive strips 2180 and 2182, respectively.

Referring to FIGS. 18, 19, 20, 21, 22, 23B and 24B, method 2400 for assembly of isolette 1700 will be further described.

At step 2404, left support frame 2010 and right support frame 2024 are attached to base subassembly 1708. Anchor pin 2014 is inserted through anchor hole 1848 and eyelet 1846 to abut anchor tab 1844. Anchor pin 2012 is inserted through anchor hole 1850 and eyelet 1852 to abut anchor tab 1854. Anchor pin 2032 is inserted through anchor hole 1824 and eyelet 1822 to abut anchor tab 1826. Anchor pin 2026 is inserted through anchor hole 1828 through eyelet 1832 to abut anchor tab 1830.

At step 2414, enclosure subassembly 1702 is positioned over left support frame 2010 and right support frame 2024, so that left support frame 2010 fits within slot 2320 and right support frame 2024 fits within slot 2362. Bottom sheet 2102 rests on base panel 1802. Base connection bay 2156 abuts left flange 1836 and back flange 1856. Base connection bay 2150 abuts right flange 1816 and back flange 1820. Front sheet 2108 abuts flange 1812.

At step 2416, drape subassembly 1704 is attached to enclosure subassembly 1702. Adhesive strip 2208 is applied along rear edge 2116, fold line 2158, and fold line 2148.

Referring to FIG. 25, a preferred method of use 2500 of an isolette as disclosed will be described.

At step 2502, the isolette is assembled as previously described. The assembled isolette is positioned on a flat surface, such as a bed, gurney, or operating table.

At step 2504, the patient is positioned inside the isolette in a supine position. Preferably, the patient's head is located adjacent the front panel and below the top panel.

At step 2506, the drape or transparent sheet is extended over the upper torso of the patient.

At step 2508, the optional flexible seal is fitted around the patient's arms and torso. The optional flexible seal is then secured in position. In a preferred embodiment, the seal is held in place with a strap attached to the table, bed, or gurney. In another preferred embodiment, the seal is held in position with medical adhesive tape.

At step 2510, the filtered negative pressure turbine is connected to the isolette through the connector.

At step 2512, pressurization is applied to the isolette. In a preferred embodiment, the isolette is capable of supporting negative pressure greater than 0.01 mmHg. In the unusual situation where a positive internal pressure is required, the isolette will support it. In this case, at this step, the positive pressure is applied.

At step 2514, medical treatment is provided to the patient through use of the access ports and optional component access panel. The preferred methods of providing medical treatment have been previously discussed.

It will be appreciated by those skilled in the art that changes could be made to the embodiments described above without departing from the broad inventive concept thereof. It is understood, therefore, that this disclosure is not limited to the particular embodiments disclosed, but it is intended to cover modifications within the spirit and scope of the present disclosure as defined by the appended claims.

1. A medical isolette comprising:
an isolette chamber constructed from a flat panel folded along a set of fold lines to create a set of isolette panels;
wherein the set of isolette panels further comprises:
a bottom panel;
a first side panel;
a second side panel;
a front panel;
a top panel; and,
a flexible drape, attached to the top panel, the first side panel and the second side panel.

2. The medical isolette of claim 1 further comprising:
a source of negative gas pressure, ductedly connected to the isolette chamber.

3. The medical isolette of claim 1 wherein:
the first side panel is connected to the bottom panel along a first fold line of the set of fold lines;
the second side panel is connected to the bottom panel along a second fold line of the set of fold lines;
the front panel is connected to the bottom panel by a third fold line of the set of fold lines; and,
the top panel is connected to the front panel by a fourth fold line of the set of fold lines.

4. The medical isolette of claim 3 wherein:
the first side panel is attached to the front panel by a first adhesive strip;
the second side panel is attached to the front panel by a second adhesive strip;
the top panel is attached to the first side panel by a third adhesive strip; and,
the top panel is attached to the second side panel by a fourth adhesive strip.

5. The medical isolette of claim 3 wherein the front panel further comprises:
an access port.

6. The medical isolette of claim 5 further comprising:
a flexible glove, sealed within the access port.

7. The medical isolette of claim 5 further comprising:
a flexible glove, removably attached to the access port.

8. The medical isolette of claim 1 wherein:
the top panel further comprises:
a first transparent window;
the first side panel further comprises a second transparent window; and,
the second side panel further comprises a third transparent window.

9. The medical isolette of claim 1 wherein the top panel slopes toward the front panel.

10. The medical isolette of claim 1 wherein the isolette chamber is formed of a semi-rigid plastic sheet.

11. A method of constructing a medical isolette comprising:
folding a first side panel toward a base panel along a first fold line;
folding a second side panel toward the base panel along a second fold line;
folding a front panel toward the base panel along a third fold line;
securing the front panel to the first side panel and the second side panel;
folding a top panel toward the first side panel and the second side panel;
securing the top panel to the first side panel and the second side panel;
attaching a flexible drape to the top panel, the first side panel and the second side panel;
connecting a set of gloves in a set of access ports resident in the front panel; and,
connecting a source of negative gas pressure to a ducted portal in one of the first side panel, the second side panel and the front panel.

12. The method of claim 11 further comprising:
providing a first transparent window in the first side panel;
providing a second transparent window in the second side panel; and,
providing a third transparent window in the top panel.

13. The method of claim 11 further comprising:
providing the top panel with a downward slope toward the front panel.

14. The method of claim 11 wherein the step of securing the front panel further comprises:
adhering the front panel to the first side panel with a first adhesive strip; and,
adhering the front panel to the second side panel with a second adhesive strip.

15. The method of claim 11 further comprising:
passing a patient respirator circuit through an access panel provided in one of the first side panel, the second side panel and the front side panel.

16. The method of claim 11 wherein the step of attaching the flexible drape further comprises:
adhering the drape to the top panel with a first adhesive strip;
adhering the drape to the first side panel with a second adhesive strip; and,
adhering the drape to the second side panel with a third adhesive strip.

17. A medical isolette comprising:
an isolette chamber constructed from a first flat panel and a second flat panel;
the first flat panel folded along a first set of fold lines to create a set of enclosure panels;
the second flat panel folded along a second set of fold lines to create a top subassembly;
wherein the set of enclosure panels further comprises:
a bottom panel;
a first side panel, adjacent the bottom panel;
a second side panel, adjacent the bottom panel; and,
a front panel, adjacent the bottom panel;
wherein the top subassembly is positioned adjacent the first side panel, the second side panel and the front panel; and,
a drape, attached to the top subassembly, the first side panel and the second side panel.

18. The medical isolette of claim 17 wherein the first flat panel further comprises:
a removable cross brace, positioned between the first side panel and the second side panel, and supporting the top subassembly.

19. The medical isolette of claim 17 wherein the top subassembly further comprises:
a window portal; and,
wherein the drape is adhered to the top panel around the window portal.

20. The medical isolette of claim 17 further comprising:
an access port positioned in one of the front panel, the first side panel and the second side panel.

21. The medical isolette of claim 20 further comprising:
a glove, positioned inside the isolette chamber, sealed within the access port.

22. The medical isolette of claim 17 further comprising:
a pair of access ports in the front panel; and,
a pair of medical gloves, positioned in a thumbs-up orientation, sealed within the pair of access ports.

23. The medical isolette of claim 17 further comprising:
a ducted pressure port, positioned in one of the first side panel, the second side panel and the front panel.

24. The medical isolette of claim 17 further comprising:
an access panel, having removable access plugs, in ducted communication with the isolette chamber.

25. The medical isolette of claim 17 wherein:
the first side panel is connected to the bottom panel along a first fold line of the first set of fold lines;
the second side panel is connected to the bottom panel along a second fold line of the first set of fold lines; and,
the front panel is connected to the bottom panel by a third fold line of the first set of fold lines.

26. The medical isolette of claim 17 wherein the top panel further comprises:
a window perimeter;
a first flap, connected to the window perimeter by a first fold line of the second set of fold lines;
a second flap, connected to the window perimeter by a second fold line of the second set of fold lines;
a third flap, connected to the window perimeter by a third fold line of the second set of fold lines; and,
a fourth flap, connected to the window perimeter by a fourth fold line of the second set of fold lines.

27. The medical isolette of claim 26 wherein:
the first flap is positioned between the first side panel and the second side panel;
the second flap is positioned adjacent the first side panel;
the third flap is positioned adjacent the front panel; and,
the fourth flap is positioned adjacent the second side panel.

28. The medical isolette of claim 26 wherein:
the first flap is connected to the window perimeter at about a 72° angle; and,
the third flap is connected to the window perimeter at about a 108° angle.

29. The medical isolette of claim 17 wherein the top panel is sloped toward the front panel.

30. A method of constructing a medical isolette comprising:
folding a first side panel toward a base panel along a first fold line;
folding a second side panel toward the base panel along a second fold line;
folding a front panel toward the base panel along a third fold line;
securing the front panel to the first side panel and the second side panel;
attaching a top panel to the first side panel, the second side panel and the front panel;
attaching a flexible drape to the top panel, the first side panel and the second side panel;
connecting a set of gloves in a set of access ports resident in the front panel; and,
connecting a source of negative gas pressure to a ducted portal in one of the first side panel, the second side panel and the front panel.

31. The method of claim 30 further comprising:
providing a window opening in the top panel; and,
covering the window opening with the drape.

32. The method of claim 30 further comprising:
providing the top panel with a downward slope toward the front panel.

33. The method of claim 30 wherein the step of securing the front panel further comprises:
adhering the front panel to the first side panel with a first adhesive strip; and,
adhering the front panel to the second side panel with a second adhesive strip.

34. The method of claim 30 further comprising:
passing a patient respirator circuit through an access panel provided in one of the first side panel, the second side panel and the front panel.

35. The method of claim 30 further comprising:
connecting a second set of gloves in a second set of access ports in one or more of the first side panel and the second side panel.

36. The method of claim 30 further comprising:
removing a cross brace from the first side panel; and,
adhering the cross brace to the first side panel and the second side panel.

37. A medical isolette comprising:
an isolette chamber constructed from a flat panel folded along a set of fold lines to create a set of isolette panels;
wherein the set of isolette panels further comprises:
a bottom panel;
a first composite side panel;
a second composite side panel;
a front panel; and,
a top panel;
a flexible drape, attached to the top panel, the first composite side panel and the second composite side panel;
a support frame subassembly positioned in the first composite side panel and the second composite side panel; and,
a base pan, adjacent the bottom panel, removably supporting the support frame subassembly.

38. The medical isolette of claim 37 further comprising:
a source of negative gas pressure ductedly connected to the isolette chamber.

39. The medical isolette of claim 37 wherein:
the top panel is connected to the front panel along a first fold line of the set of fold lines;
the bottom panel is connected to the front panel along a second fold line of the set of fold lines;
the first composite side panel is connected to the front panel along a third fold line of the set of fold lines; and,
the second composite side panel is connected to the front panel along a third fold line of the set of fold lines.

40. The medical isolette of claim 37:
wherein the first composite side panel is further comprised of a first exterior panel and a first interior panel separated by a first slot;
wherein the second composite side panel is further comprised of a second exterior panel and a second interior panel separated by a second slot;
wherein the support frame subassembly further comprises a first wire frame and a second wire frame; and,
wherein the first wire frame is positioned within the first slot and the second wire frame is positioned within the second slot.

41. The medical isolette of claim 40:
wherein the first exterior side panel is connected to the first interior side panel by a first fold line of the set of fold lines; and,
wherein the second exterior side panel is connected to the second interior side panel by a second fold line of the set of fold lines.

42. The medical isolette of claim 41 wherein:
the first side panel is fixed to the front panel by a first adhesive strip;
the second side panel is fixed to the front panel by a second adhesive strip;
the first side panel is attached to the base panel by a third adhesive strip;
the second side panel is attached to the base panel by a fourth adhesive strip;
the first side panel is attached to the top panel by a fifth adhesive strip; and,
the second side panel is attached to the top panel by a sixth adhesive strip.

43. The medical isolette of claim 40 wherein:
the base pan is connected to the first wire frame by a first set of 3-point connections; and,
the base pan is connected to the second wire frame by a second set of 3-point connections.

44. The medical isolette of claim 37 wherein the top panel further comprises:
an observation window.

45. The medical isolette of claim 37:
wherein the flat panel is constructed of a plastic material; and,
wherein the base pan is constructed of a metallic material.

46. The medical isolette of claim 37 further comprising:
a set of access ports, fixed in at least one of the front panel, the first composite side panel and the second composite side panel; and,
a set of medical gloves, sealed into the set of access panels, projecting into the isolette chamber.

47. The medical isolette of claim 46 wherein the set of medical gloves is removable.

48. The medical isolette of claim 37 further comprising:
an access panel; and,
a set of sealable portals, in the access panel, ductedly connected to the isolette chamber.

49. The medical isolette of claim 37 wherein the top panel slopes toward the front panel.

50. A method of constructing a medical isolette comprising;
folding a first exterior panel toward a first interior panel to create a first slot in a first composite panel;
folding the first composite panel toward a front panel;
fixing the first composite panel to the front panel;
folding a second exterior panel toward a second internal panel to create a second slot in a second composite panel;
folding the second composite panel toward the front panel;
fixing the second composite panel to the front panel folding a bottom sheet toward the front sheet until adjacent the first composite panel and the second composite panel;
fixing the bottom sheet to the first composite panel and the second composite panel;
folding a top sheet toward the front sheet until adjacent the first composite panel and the second composite panel;
fixing the top sheet to the first composite panel and the second composite panel;
positioning a first support frame and a second support frame in a base pan;
positioning the first support frame in the first slot and the second support frame in the second slot; and,
fixing a drape subassembly to the top panel, the first composite panel and the second composite panel.

51. The method of claim 50 further comprising:
providing an access port in at least one of the front panel, the first composite panel and the second composite panel; and,
fixing a medical glove into the access port.

52. The method of claim 50 further comprising:
ductedly connecting a negative gas pressure source through at least one of the front panel, the first composite panel and the second composite panel.

53. The method of claim 50 further comprising:
positioning a transparent sealed window in the top panel.

54. The method of claim 50 further comprising:
positioning an access panel in at least one of the front panel, the first composite panel and the second composite panel.

55. The method of claim 50 further comprising:
providing a slope in the top panel toward the front panel.
